SEQUENCE LISTING (343 sequences)

SEQ ID NO: 1 - DNA, 23 bp, Homo sapiens
aagggcgtgttcgtgctgaataa

SEQ ID NO: 2 - DNA, 23 bp, Homo sapiens
aaggctgccgtctaccatcattt

SEQ ID NO: 3 - DNA, 23 bp, Homo sapiens
aacggctgaagcacctcattgtg

SEQ ID NO: 4 - DNA, 23 bp, Homo sapiens
aagcaggactccttgtcttctca

SEQ ID NO: 5 - DNA, 23 bp, Homo sapiens
aaccagcaccggaaacagaaaag

SEQ ID NO: 6 - DNA, 23 bp, Homo sapiens
aagtggagggaactgcctttgtc

SEQ ID NO: 7 - DNA, 23 bp, Homo sapiens
aagggcctggaggtcaccatcac

SEQ ID NO: 8 - DNA, 23 bp, Homo sapiens
aagcccaacctcagctacatcat

SEQ ID NO: 9 - DNA, 23 bp, Homo sapiens
aagcaggagacttccttgaagcc

SEQ ID NO: 10 - DNA, 23 bp, Homo sapiens
aatgccctttgacctcatggtgt

SEQ ID NO: 11 - DNA, 23 bp, Homo sapiens
aagatcaactcacctgtaataaa

SEQ ID NO: 12 - DNA, 23 bp, Homo sapiens
aaggcctgtgagccaggagtgga

SEQ ID NO: 13 - DNA, 23 bp, Homo sapiens
aatccgagccgttctctacaatt

SEQ ID NO: 14 - DNA, 23 bp, Homo sapiens
aagcgcattccgattgaggatgg

SEQ ID NO: 15 - DNA, 23 bp, Homo sapiens
aaggtcgtgccggaaggaatcag

SEQ ID NO: 16 - DNA, 23 bp, Homo sapiens
aagaccggcctgcaggaagtgga

SEQ ID NO: 17 - DNA, 23 bp, Homo sapiens
aagctggaggagaagaaacacta

SEQ ID NO: 18 - DNA, 23 bp, Homo sapiens
aatggacaaagtcggcaagtacc

SEQ ID NO: 19 - DNA, 23 bp, Homo sapiens
aaggaggatggaaagctgaacaa

SEQ ID NO: 20 - DNA, 23 bp, Homo sapiens
aagaggcctcaggatgccaagaa

SEQ ID NO: 21 - DNA, 23 bp, Homo sapiens
aacagggagttcaagtcagaaaa

SEQ ID NO: 22 - DNA, 23 bp, Homo sapiens
aagacacccaagtacttcaaacc

SEQ ID NO: 23 - DNA, 23 bp, Homo sapiens
aagatccgagcctactatgaaaa

SEQ ID NO: 24 - DNA, 23 bp, Homo sapiens
aagccttggctcaataccaaaag

SEQ ID NO: 25 - DNA, 23 bp, Homo sapiens
aagctctgccgtgatgaactgtg

SEQ ID NO: 26 - RNA, 25 nt, Artificial Sequence
  Description of Artificial Sequence Synthetic oligonucleotide
caaguccucguuguccguuccauau

SEQ ID NO: 27 - RNA, 25 nt, Artificial Sequence
  Description of Artificial Sequence Synthetic oligonucleotide
aggcugccgucuaccaucauuucau

SEQ ID NO: 28 - RNA, 25 nt, Artificial Sequence
  Description of Artificial Sequence Synthetic oligonucleotide
caucucgcugcaggaggcuaaagau

SEQ ID NO: 29 - RNA, 25 nt, Artificial Sequence
  Description of Artificial Sequence Synthetic oligonucleotide
ggccaaagaucaacucaccuguaau

SEQ ID NO: 30 - RNA, 25 nt, Artificial Sequence
  Description of Artificial Sequence Synthetic oligonucleotide
ccaagaacacuaugauccuugagau

SEQ ID NO: 31 - RNA, 25 nt, Artificial Sequence
  Description of Artificial Sequence Synthetic oligonucleotide
cauauccaugaugacuggcuuugcu

SEQ ID NO: 32 - RNA, 25 nt, Artificial Sequence
  Description of Artificial Sequence Synthetic oligonucleotide
gccaaugguguugacagauacaucu

SEQ ID NO: 33 - RNA, 25 nt, Artificial Sequence
  Description of Artificial Sequence Synthetic oligonucleotide
gagcuggacaaagccuucuccgaua

SEQ ID NO: 34 - RNA, 25 nt, Artificial Sequence
  Description of Artificial Sequence Synthetic oligonucleotide
gagccaggaguggacuauguguaca

SEQ ID NO: 35 - RNA, 25 nt, Artificial Sequence
  Description of Artificial Sequence Synthetic oligonucleotide
ccuucaccgagagcaugguugucuu

SEQ ID NO: 36 - DNA, 23 bp, Homo sapiens
aaccgccagcggaagatcaagaa

SEQ ID NO: 37 - DNA, 23 bp, Homo sapiens
aaccggaaggtgtatgaactgag

SEQ ID NO: 38 - DNA, 23 bp, Homo sapiens
aagggctggagctgtttgagaac

SEQ ID NO: 39 - DNA, 23 bp, Homo sapiens
aattcccagcagactccaatgtg

SEQ ID NO: 40 - DNA, 23 bp, Homo sapiens
aatgggcactgcaggcctcagca

SEQ ID NO: 41 - DNA, 23 bp, Homo sapiens
aaggtgtatgaactgagcaatgt

SEQ ID NO: 42 - DNA, 23 bp, Homo sapiens
aagggaccgaggtgacagtgaag

SEQ ID NO: 43 - DNA, 23 bp, Homo sapiens
aagaagatagccaaccaatgtgc

SEQ ID NO: 44 - DNA, 23 bp, Homo sapiens
aaccagacccgggagcttcgtgt

SEQ ID NO: 45 - DNA, 23 bp, Homo sapiens
aacccattgcccgagctcaagtg

SEQ ID NO: 46 - DNA, 23 bp, Homo sapiens
aactgccctgatgggcagtcaac

SEQ ID NO: 47 - DNA, 23 bp, Homo sapiens
aagccagaggtctcagaagggac

SEQ ID NO: 48 - DNA, 23 bp, Homo sapiens
aacaaccggaaggtgtatgaact

SEQ ID NO: 49 - DNA, 23 bp, Homo sapiens
aaggcctcagtcagtgtgaccgc

SEQ ID NO: 50 - DNA, 23 bp, Homo sapiens
aaccaatgtgctattcaaactgc

SEQ ID NO: 51 - DNA, 23 bp, Homo sapiens
aatgcccagacatctgtgtcccc

SEQ ID NO: 52 - DNA, 23 bp, Homo sapiens
aagtgtgaggcccaccctagagc

SEQ ID NO: 53 - DNA, 23 bp, Homo sapiens
aaccagagccaggagacactgca

SEQ ID NO: 54 - DNA, 23 bp, Homo sapiens
aactgagcaatgtgcaagaagat

SEQ ID NO: 55 - RNA, 25 nt, Artificial Sequence
  Description of Artificial Sequence Synthetic oligonucleotide
gagcaaugugcaagaagauagccaa

SEQ ID NO: 56 - RNA, 25 nt, Artificial Sequence
  Description of Artificial Sequence Synthetic oligonucleotide
gauagccaaccaaugugcuauucaa

SEQ ID NO: 57 - RNA, 25 nt, Artificial Sequence
  Description of Artificial Sequence Synthetic oligonucleotide
cccugaugggcagucaacagcuaaa

SEQ ID NO: 58 - RNA, 25 nt, Artificial Sequence
  Description of Artificial Sequence Synthetic oligonucleotide
acugugguagcagccgcagucauaa

SEQ ID NO: 59 - RNA, 25 nt, Artificial Sequence
  Description of Artificial Sequence Synthetic oligonucleotide
caggccucagcacguaccucuauaa

SEQ ID NO: 60 - RNA, 25 nt, Artificial Sequence
  Description of Artificial Sequence Synthetic oligonucleotide
ccacacugaacagaguggaagacau

SEQ ID NO: 61 - RNA, 25 nt, Artificial Sequence
  Description of Artificial Sequence Synthetic oligonucleotide
gcauuguccucagucagauacaaca

SEQ ID NO: 62 - RNA, 25 nt, Artificial Sequence
  Description of Artificial Sequence Synthetic oligonucleotide
caucugaucuguagucacaugacua

SEQ ID NO: 63 - RNA, 25 nt, Artificial Sequence
  Description of Artificial Sequence Synthetic oligonucleotide
gaggaaggagcaagacucaagacau

SEQ ID NO: 64 - RNA, 25 nt, Artificial Sequence
  Description of Artificial Sequence Synthetic oligonucleotide
ggacauacaacugggaaauacugaa

SEQ ID NO: 65 - DNA, 23 bp, Homo sapiens
aagggcgtgttcgtgctgaataa

SEQ ID NO: 66 - DNA, 23 bp, Homo sapiens
aaggctgccgtctaccatcattt

SEQ ID NO: 67 - DNA, 23 bp, Homo sapiens
aacggctgaagcacctcattgtg

SEQ ID NO: 68 - DNA, 23 bp, Homo sapiens
aagcaggactccttgtcttctca

SEQ ID NO: 69 - DNA, 23 bp, Homo sapiens
aaccagcaccggaaacagaaaag

SEQ ID NO: 70 - DNA, 23 bp, Homo sapiens
aagtggagggaactgcctttgtc

SEQ ID NO: 71 - DNA, 23 bp, Homo sapiens
aagggcctggaggtcaccatcac

SEQ ID NO: 72 - DNA, 23 bp, Homo sapiens
aagcccaacctcagctacatcat

SEQ ID NO: 73 - DNA, 23 bp, Homo sapiens
aagcaggagacttccttgaagcc

SEQ ID NO: 74 - DNA, 23 bp, Homo sapiens
aatgccctttgacctcatggtgt

SEQ ID NO: 75 - DNA, 23 bp, Homo sapiens
aagatcaactcacctgtaataaa

SEQ ID NO: 76 - DNA, 23 bp, Homo sapiens
aaggcctgtgagccaggagtgga

SEQ ID NO: 77 - DNA, 23 bp, Homo sapiens
aatccgagccgttctctacaatt

SEQ ID NO: 78 - DNA, 23 bp, Homo sapiens
aagcgcattccgattgaggatgg

SEQ ID NO: 79 - DNA, 23 bp, Homo sapiens
aaggtcgtgccggaaggaatcag

SEQ ID NO: 80 - DNA, 23 bp, Homo sapiens
aagaccggcctgcaggaagtgga

SEQ ID NO: 81 - DNA, 23 bp, Homo sapiens
aagctggaggagaagaaacacta

SEQ ID NO: 82 - DNA, 23 bp, Homo sapiens
aatggacaaagtcggcaagtacc

SEQ ID NO: 83 - DNA, 23 bp, Homo sapiens
aaggaggatggaaagctgaacaa

SEQ ID NO: 84 - DNA, 23 bp, Homo sapiens
aagaggcctcaggatgccaagaa

SEQ ID NO: 85 - DNA, 23 bp, Homo sapiens
aacagggagttcaagtcagaaaa

SEQ ID NO: 86 - DNA, 23 bp, Homo sapiens
aagacacccaagtacttcaaacc

SEQ ID NO: 87 - DNA, 23 bp, Homo sapiens
aagatccgagcctactatgaaaa

SEQ ID NO: 88 - DNA, 23 bp, Homo sapiens
aagccttggctcaataccaaaag

SEQ ID NO: 89 - DNA, 23 bp, Homo sapiens
aagctctgccgtgatgaactgtg

SEQ ID NO: 90 - RNA, 25 nt, Artificial Sequence
  Description of Artificial Sequence Synthetic oligonucleotide
cgugauccuuggagccucaaauaua

SEQ ID NO: 91 - RNA, 25 nt, Artificial Sequence
  Description of Artificial Sequence Synthetic oligonucleotide
cagaaucuagauaucuugcucagau

SEQ ID NO: 92 - RNA, 25 nt, Artificial Sequence
  Description of Artificial Sequence Synthetic oligonucleotide
gcucagauuggugacuccgucucau

SEQ ID NO: 93 - RNA, 25 nt, Artificial Sequence
  Description of Artificial Sequence Synthetic oligonucleotide
gaacccagauagauaguccacugaa

SEQ ID NO: 94 - RNA, 25 nt, Artificial Sequence
  Description of Artificial Sequence Synthetic oligonucleotide
ggaauccagguggagaucuacucuu

SEQ ID NO: 95 - RNA, 25 nt, Artificial Sequence
  Description of Artificial Sequence Synthetic oligonucleotide
caagaguuuggaaguaaccuuuacu

SEQ ID NO: 96 - RNA, 25 nt, Artificial Sequence
  Description of Artificial Sequence Synthetic oligonucleotide
ugcccacaguaaggcaggcuguaaa

SEQ ID NO: 97 - RNA, 25 nt, Artificial Sequence
  Description of Artificial Sequence Synthetic oligonucleotide
aagcauucccuagagauccagaaau

SEQ ID NO: 98 - RNA, 25 nt, Artificial Sequence
  Description of Artificial Sequence Synthetic oligonucleotide
gaaggagacacugucaucaucucuu

SEQ ID NO: 99 - RNA, 25 nt, Artificial Sequence
  Description of Artificial Sequence Synthetic oligonucleotide
gcaaauccuugauacugcucaucau

SEQ ID NO: 100 - RNA, 19 nt, Artificial Sequence
  Description of Artificial Sequence Synthetic oligonucleotide
ucaucugaagaucagcuau

SEQ ID NO: 101 - RNA, 19 nt, Artificial Sequence
  Description of Artificial Sequence Synthetic oligonucleotide
ccuuuggaccugaucagcu

SEQ ID NO: 102 - RNA, 19 nt, Artificial Sequence
  Description of Artificial Sequence Synthetic oligonucleotide
gcugacuaauuauucggua

SEQ ID NO: 103 - RNA, 19 nt, Artificial Sequence
  Description of Artificial Sequence Synthetic oligonucleotide
ccaacgcaaagcaauacau

SEQ ID NO: 104 - RNA, 19 nt, Artificial Sequence
  Description of Artificial Sequence Synthetic oligonucleotide
gcaucccaguaaugguugu

SEQ ID NO: 105 - RNA, 19 nt, Artificial Sequence
  Description of Artificial Sequence Synthetic oligonucleotide
ucccauggguuguguguuu

SEQ ID NO: 106 - RNA, 19 nt, Artificial Sequence
  Description of Artificial Sequence Synthetic oligonucleotide
ccauggguuguguguuuau

SEQ ID NO: 107 - RNA, 19 nt, Artificial Sequence
  Description of Artificial Sequence Synthetic oligonucleotide
gcaaucugagccagugcuu

SEQ ID NO: 108 - RNA, 19 nt, Artificial Sequence
  Description of Artificial Sequence Synthetic oligonucleotide
gccagugcuuuaauggcau

SEQ ID NO: 109 - RNA, 19 nt, Artificial Sequence
  Description of Artificial Sequence Synthetic oligonucleotide
gcuuccaaauauuguugac

SEQ ID NO: 110 - RNA, 25 nt, Artificial Sequence
  Description of Artificial Sequence Synthetic oligonucleotide
gaucaucugaagaucagcuauuaga

SEQ ID NO: 111 - RNA, 25 nt, Artificial Sequence
  Description of Artificial Sequence Synthetic oligonucleotide
caguuaaguccuuuggaccugauca

SEQ ID NO: 112 - RNA, 25 nt, Artificial Sequence
  Description of Artificial Sequence Synthetic oligonucleotide
uaacugacuugaauguccaacgcaa

SEQ ID NO: 113 - RNA, 25 nt, Artificial Sequence
  Description of Artificial Sequence Synthetic oligonucleotide
cgaggucgaagagcaucccaguaau

SEQ ID NO: 114 - RNA, 25 nt, Artificial Sequence
  Description of Artificial Sequence Synthetic oligonucleotide
caguaaugguuguccugccugcaau

SEQ ID NO: 115 - RNA, 25 nt, Artificial Sequence
  Description of Artificial Sequence Synthetic oligonucleotide
aaugguuguccugccugcaauauuu

SEQ ID NO: 116 - RNA, 25 nt, Artificial Sequence
  Description of Artificial Sequence Synthetic oligonucleotide
gcaaggcuaugugauuacaaggcuu

SEQ ID NO: 117 - RNA, 25 nt, Artificial Sequence
  Description of Artificial Sequence Synthetic oligonucleotide
caagaucccauggguuguguguuua

SEQ ID NO: 118 - RNA, 25 nt, Artificial Sequence
  Description of Artificial Sequence Synthetic oligonucleotide
ggguuguguguuuauuucacuugau

SEQ ID NO: 119 - RNA, 25 nt, Artificial Sequence
  Description of Artificial Sequence Synthetic oligonucleotide
ccugcaaucugagccagugcuuuaa

SEQ ID NO: 120 - RNA, 19 nt, Artificial Sequence
  Description of Artificial Sequence Synthetic oligonucleotide
gcaagucccagauauacua

SEQ ID NO: 121 - RNA, 19 nt, Artificial Sequence
  Description of Artificial Sequence Synthetic oligonucleotide
gcccaaguuugaaggacaa

SEQ ID NO: 122 - RNA, 19 nt, Artificial Sequence
  Description of Artificial Sequence Synthetic oligonucleotide
cccaaguuugaaggacaaa

SEQ ID NO: 123 - RNA, 19 nt, Artificial Sequence
  Description of Artificial Sequence Synthetic oligonucleotide
ccugguguggugugguuua

SEQ ID NO: 124 - RNA, 19 nt, Artificial Sequence
  Description of Artificial Sequence Synthetic oligonucleotide
ucaguaggaguuucuggaa

SEQ ID NO: 125 - RNA, 19 nt, Artificial Sequence
  Description of Artificial Sequence Synthetic oligonucleotide
ggaguuucuggaaaccuau

SEQ ID NO: 126 - RNA, 19 nt, Artificial Sequence
  Description of Artificial Sequence Synthetic oligonucleotide
ggaaaccuaucuuuaccaa

SEQ ID NO: 127 - RNA, 19 nt, Artificial Sequence
  Description of Artificial Sequence Synthetic oligonucleotide
ccacugaaagagugacuuu

SEQ ID NO: 128 - RNA, 19 nt, Artificial Sequence
  Description of Artificial Sequence Synthetic oligonucleotide
gaagagauccuucuguaaa

SEQ ID NO: 129 - RNA, 19 nt, Artificial Sequence
  Description of Artificial Sequence Synthetic oligonucleotide
ggaauuaugucugcugaau

SEQ ID NO: 130 - RNA, 25 nt, Artificial Sequence
  Description of Artificial Sequence Synthetic oligonucleotide
aagucccagauauacuacaacucaa

SEQ ID NO: 131 - RNA, 25 nt, Artificial Sequence
  Description of Artificial Sequence Synthetic oligonucleotide
gcccaaguuugaaggacaaaccgaa

SEQ ID NO: 132 - RNA, 25 nt, Artificial Sequence
  Description of Artificial Sequence Synthetic oligonucleotide
cagcccugguguggugugguuuaaa

SEQ ID NO: 133 - RNA, 25 nt, Artificial Sequence
  Description of Artificial Sequence Synthetic oligonucleotide
cccugguguggugugguuuaaagau

SEQ ID NO: 134 - RNA, 25 nt, Artificial Sequence
  Description of Artificial Sequence Synthetic oligonucleotide
ugguguggugugguuuaaagauuca

SEQ ID NO: 135 - RNA, 25 nt, Artificial Sequence
  Description of Artificial Sequence Synthetic oligonucleotide
ucaguaggaguuucuggaaaccuau

SEQ ID NO: 136 - RNA, 25 nt, Artificial Sequence
  Description of Artificial Sequence Synthetic oligonucleotide
uaggaguuucuggaaaccuaucuuu

SEQ ID NO: 137 - RNA, 25 nt, Artificial Sequence
  Description of Artificial Sequence Synthetic oligonucleotide
aggaguuucuggaaaccuaucuuua

SEQ ID NO: 138 - RNA, 25 nt, Artificial Sequence
  Description of Artificial Sequence Synthetic oligonucleotide
cccaccacugaaagagugacuuuga

SEQ ID NO: 139 - RNA, 25 nt, Artificial Sequence
  Description of Artificial Sequence Synthetic oligonucleotide
caccacugaaagagugacuuugaca

SEQ ID NO: 140 - RNA, 19 nt, Artificial Sequence
  Description of Artificial Sequence Synthetic oligonucleotide
gcaucucagcccugagaaa

SEQ ID NO: 141 - RNA, 19 nt, Artificial Sequence
  Description of Artificial Sequence Synthetic oligonucleotide
gcccugagaaaggagacau

SEQ ID NO: 142 - RNA, 19 nt, Artificial Sequence
  Description of Artificial Sequence Synthetic oligonucleotide
ggaugcuuccaaucuggau

SEQ ID NO: 143 - RNA, 19 nt, Artificial Sequence
  Description of Artificial Sequence Synthetic oligonucleotide
gcuuccaaucuggauucaa

SEQ ID NO: 144 - RNA, 19 nt, Artificial Sequence
  Description of Artificial Sequence Synthetic oligonucleotide
ggauucaaugaggagacuu

SEQ ID NO: 145 - RNA, 19 nt, Artificial Sequence
  Description of Artificial Sequence Synthetic oligonucleotide
gcaggacaugacaacucau

SEQ ID NO: 146 - RNA, 19 nt, Artificial Sequence
  Description of Artificial Sequence Synthetic oligonucleotide
ggcaccucagauuguuguu

SEQ ID NO: 147 - RNA, 19 nt, Artificial Sequence
  Description of Artificial Sequence Synthetic oligonucleotide
ccucagauuguuguuguua

SEQ ID NO: 148 - RNA, 19 nt, Artificial Sequence
  Description of Artificial Sequence Synthetic oligonucleotide
gcacagaacuuauguuguu

SEQ ID NO: 149 - RNA, 19 nt, Artificial Sequence
  Description of Artificial Sequence Synthetic oligonucleotide
ggaaaguggcuaugcaguu

SEQ ID NO: 150 - RNA, 25 nt, Artificial Sequence
  Description of Artificial Sequence Synthetic oligonucleotide
cagcccugagaaaggagacauguaa

SEQ ID NO: 151 - RNA, 25 nt, Artificial Sequence
  Description of Artificial Sequence Synthetic oligonucleotide
uggaugcuuccaaucuggauucaau

SEQ ID NO: 152 - RNA, 25 nt, Artificial Sequence
  Description of Artificial Sequence Synthetic oligonucleotide
gagguauaccuagaguaccuccaga

SEQ ID NO: 153 - RNA, 25 nt, Artificial Sequence
  Description of Artificial Sequence Synthetic oligonucleotide
ccuccagaacagauuugagaguagu

SEQ ID NO: 154 - RNA, 25 nt, Artificial Sequence
  Description of Artificial Sequence Synthetic oligonucleotide
caacucaucucauucugcgcagcuu

SEQ ID NO: 155 - RNA, 25 nt, Artificial Sequence
  Description of Artificial Sequence Synthetic oligonucleotide
gggcaccucagauuguuguuguuaa

SEQ ID NO: 156 - RNA, 25 nt, Artificial Sequence
  Description of Artificial Sequence Synthetic oligonucleotide
cacugggcacagaacuuauguuguu

SEQ ID NO: 157 - RNA, 25 nt, Artificial Sequence
  Description of Artificial Sequence Synthetic oligonucleotide
ggcacagaacuuauguuguucucua

SEQ ID NO: 158 - RNA, 25 nt, Artificial Sequence
  Description of Artificial Sequence Synthetic oligonucleotide
gcacagaacuuauguuguucucuau

SEQ ID NO: 159 - RNA, 25 nt, Artificial Sequence
  Description of Artificial Sequence Synthetic oligonucleotide
uggaaaguguaggcuuaccucaaau

SEQ ID NO: 160 - RNA, 19 nt, Artificial Sequence
  Description of Artificial Sequence Synthetic oligonucleotide
acucccagucuugucauug

SEQ ID NO: 161 - RNA, 19 nt, Artificial Sequence
  Description of Artificial Sequence Synthetic oligonucleotide
cccagucuugucauugcca

SEQ ID NO: 162 - RNA, 19 nt, Artificial Sequence
  Description of Artificial Sequence Synthetic oligonucleotide
ccagucuugucauugccag

SEQ ID NO: 163 - RNA, 19 nt, Artificial Sequence
  Description of Artificial Sequence Synthetic oligonucleotide
gcuguguugguagugcugu

SEQ ID NO: 164 - RNA, 19 nt, Artificial Sequence
  Description of Artificial Sequence Synthetic oligonucleotide
gguagugcuguguugaauu

SEQ ID NO: 165 - RNA, 19 nt, Artificial Sequence
  Description of Artificial Sequence Synthetic oligonucleotide
guagugcuguguugaauua

SEQ ID NO: 166 - RNA, 19 nt, Artificial Sequence
  Description of Artificial Sequence Synthetic oligonucleotide
gcuguguugaauuacggaa

SEQ ID NO: 167 - RNA, 19 nt, Artificial Sequence
  Description of Artificial Sequence Synthetic oligonucleotide
cuguguugaauuacggaau

SEQ ID NO: 168 - RNA, 19 nt, Artificial Sequence
  Description of Artificial Sequence Synthetic oligonucleotide
acuccacagucaauauuag

SEQ ID NO: 169 - RNA, 25 nt, Artificial Sequence
  Description of Artificial Sequence Synthetic oligonucleotide
gcuguguugguagugcuguguugaa

SEQ ID NO: 170 - RNA, 25 nt, Artificial Sequence
  Description of Artificial Sequence Synthetic oligonucleotide
uguguugguagugcuguguugaauu

SEQ ID NO: 171 - RNA, 25 nt, Artificial Sequence
  Description of Artificial Sequence Synthetic oligonucleotide
gguagugcuguguugaauuacggaa

SEQ ID NO: 172 - RNA, 25 nt, Artificial Sequence
  Description of Artificial Sequence Synthetic oligonucleotide
uagugcuguguugaauuacggaaua

SEQ ID NO: 173 - RNA, 25 nt, Artificial Sequence
  Description of Artificial Sequence Synthetic oligonucleotide
agugcuguguugaauuacggaauaa

SEQ ID NO: 174 - RNA, 25 nt, Artificial Sequence
  Description of Artificial Sequence Synthetic oligonucleotide
gcuguguugaauuacggaauaauga

SEQ ID NO: 175 - RNA, 19 nt, Artificial Sequence
  Description of Artificial Sequence Synthetic oligonucleotide
ggacaccaugagcacugaa

SEQ ID NO: 176 - RNA, 19 nt, Artificial Sequence
  Description of Artificial Sequence Synthetic oligonucleotide
ccaugagcacugaaagcau

SEQ ID NO: 177 - RNA, 19 nt, Artificial Sequence
  Description of Artificial Sequence Synthetic oligonucleotide
gccuguagcccauguugua

SEQ ID NO: 178 - RNA, 19 nt, Artificial Sequence
  Description of Artificial Sequence Synthetic oligonucleotide
gcguggagcugagagauaa

SEQ ID NO: 179 - RNA, 19 nt, Artificial Sequence
  Description of Artificial Sequence Synthetic oligonucleotide
gcccgacuaucucgacuuu

SEQ ID NO: 180 - RNA, 19 nt, Artificial Sequence
  Description of Artificial Sequence Synthetic oligonucleotide
cccaagcuuagaacuuuaa

SEQ ID NO: 181 - RNA, 19 nt, Artificial Sequence
  Description of Artificial Sequence Synthetic oligonucleotide
gcuggcaaccacuaagaau

SEQ ID NO: 182 - RNA, 19 nt, Artificial Sequence
  Description of Artificial Sequence Synthetic oligonucleotide
gcaaccacuaagaauucaa

SEQ ID NO: 183 - RNA, 19 nt, Artificial Sequence
  Description of Artificial Sequence Synthetic oligonucleotide
gccagcucccucuauuuau

SEQ ID NO: 184 - RNA, 19 nt, Artificial Sequence
  Description of Artificial Sequence Synthetic oligonucleotide
gcucccucuauuuauguuu

SEQ ID NO: 185 - RNA, 25 nt, Artificial Sequence
  Description of Artificial Sequence Synthetic oligonucleotide
uggagucgugcauaggacuugcaaa

SEQ ID NO: 186 - RNA, 25 nt, Artificial Sequence
  Description of Artificial Sequence Synthetic oligonucleotide
gaucauugcccuauccgaauaucuu

SEQ ID NO: 187 - RNA, 25 nt, Artificial Sequence
  Description of Artificial Sequence Synthetic oligonucleotide
cccuauccgaauaucuuccugugau

SEQ ID NO: 188 - RNA, 25 nt, Artificial Sequence
  Description of Artificial Sequence Synthetic oligonucleotide
gaaccagccuuuagugccuaccauu

SEQ ID NO: 189 - RNA, 25 nt, Artificial Sequence
  Description of Artificial Sequence Synthetic oligonucleotide
cagccuuuagugccuaccauuaucu

SEQ ID NO: 190 - RNA, 25 nt, Artificial Sequence
  Description of Artificial Sequence Synthetic oligonucleotide
ccuuuagugccuaccauuaucuuau

SEQ ID NO: 191 - RNA, 25 nt, Artificial Sequence
  Description of Artificial Sequence Synthetic oligonucleotide
gacaaagaucuugccuuacagacuu

SEQ ID NO: 192 - RNA, 25 nt, Artificial Sequence
  Description of Artificial Sequence Synthetic oligonucleotide
gauucuguaacugcagacuucauua

SEQ ID NO: 193 - RNA, 25 nt, Artificial Sequence
  Description of Artificial Sequence Synthetic oligonucleotide
ucuguaacugcagacuucauuagca

SEQ ID NO: 194 - RNA, 25 nt, Artificial Sequence
  Description of Artificial Sequence Synthetic oligonucleotide
cagacuucauuagcacacagauuca

SEQ ID NO: 195 - RNA, 19 nt, Artificial Sequence
  Description of Artificial Sequence Synthetic oligonucleotide
ccaaguguccauaccucaa

SEQ ID NO: 196 - RNA, 19 nt, Artificial Sequence
  Description of Artificial Sequence Synthetic oligonucleotide
ggucuuucucacuucuguu

SEQ ID NO: 197 - RNA, 19 nt, Artificial Sequence
  Description of Artificial Sequence Synthetic oligonucleotide
gcuguccuguggucacaau

SEQ ID NO: 198 - RNA, 19 nt, Artificial Sequence
  Description of Artificial Sequence Synthetic oligonucleotide
gggcacauacgaguguguu

SEQ ID NO: 199 - RNA, 19 nt, Artificial Sequence
  Description of Artificial Sequence Synthetic oligonucleotide
gcugacuucccuacaccua

SEQ ID NO: 200 - RNA, 19 nt, Artificial Sequence
  Description of Artificial Sequence Synthetic oligonucleotide
gcagcaaacuggauuucaa

SEQ ID NO: 201 - RNA, 19 nt, Artificial Sequence
  Description of Artificial Sequence Synthetic oligonucleotide
gcuuugcaggaagugucua

SEQ ID NO: 202 - RNA, 19 nt, Artificial Sequence
  Description of Artificial Sequence Synthetic oligonucleotide
gcugcuggaaguagaauuu

SEQ ID NO: 203 - RNA, 19 nt, Artificial Sequence
  Description of Artificial Sequence Synthetic oligonucleotide
ggaaguagaauuuguccaa

SEQ ID NO: 204 - RNA, 19 nt, Artificial Sequence
  Description of Artificial Sequence Synthetic oligonucleotide
ggucaacuucagagacuau

SEQ ID NO: 205 - RNA, 25 nt, Artificial Sequence
  Description of Artificial Sequence Synthetic oligonucleotide
gagcuggcacaaacucgcaucuacu

SEQ ID NO: 206 - RNA, 25 nt, Artificial Sequence
  Description of Artificial Sequence Synthetic oligonucleotide
gggacaugaauauauggcccgagua

SEQ ID NO: 207 - RNA, 25 nt, Artificial Sequence
  Description of Artificial Sequence Synthetic oligonucleotide
cggaccaucuuugauaucacuaaua

SEQ ID NO: 208 - RNA, 25 nt, Artificial Sequence
  Description of Artificial Sequence Synthetic oligonucleotide
gcacauacgaguguguuguucugaa

SEQ ID NO: 209 - RNA, 25 nt, Artificial Sequence
  Description of Artificial Sequence Synthetic oligonucleotide
ggagaagaauuaaaugccaucaaca

SEQ ID NO: 210 - RNA, 25 nt, Artificial Sequence
  Description of Artificial Sequence Synthetic oligonucleotide
gaagggaaaguguacgcccuguaua

SEQ ID NO: 211 - RNA, 25 nt, Artificial Sequence
  Description of Artificial Sequence Synthetic oligonucleotide
ccuccauuugcaauugaccucuucu

SEQ ID NO: 212 - RNA, 25 nt, Artificial Sequence
  Description of Artificial Sequence Synthetic oligonucleotide
gaacuuccucagauggacaagauua

SEQ ID NO: 213 - RNA, 25 nt, Artificial Sequence
  Description of Artificial Sequence Synthetic oligonucleotide
cagauuuccuaacucuggugcucuu

SEQ ID NO: 214 - RNA, 25 nt, Artificial Sequence
  Description of Artificial Sequence Synthetic oligonucleotide
aggaaguauggcaugaacaucuuua

SEQ ID NO: 215 - RNA, 19 nt, Artificial Sequence
  Description of Artificial Sequence Synthetic oligonucleotide
gcugcuguaacagggacua

SEQ ID NO: 216 - RNA, 19 nt, Artificial Sequence
  Description of Artificial Sequence Synthetic oligonucleotide
gcacuaugggacugaguaa

SEQ ID NO: 217 - RNA, 19 nt, Artificial Sequence
  Description of Artificial Sequence Synthetic oligonucleotide
ccucugaagauucaagcuu

SEQ ID NO: 218 - RNA, 19 nt, Artificial Sequence
  Description of Artificial Sequence Synthetic oligonucleotide
ccugagacuucacaaucuu

SEQ ID NO: 219 - RNA, 19 nt, Artificial Sequence
  Description of Artificial Sequence Synthetic oligonucleotide
ggacaagggcuuguaucaa

SEQ ID NO: 220 - RNA, 19 nt, Artificial Sequence
  Description of Artificial Sequence Synthetic oligonucleotide
ccacaggaaugauucgcau

SEQ ID NO: 221 - RNA, 19 nt, Artificial Sequence
  Description of Artificial Sequence Synthetic oligonucleotide
gcucaucuauacacgguua

SEQ ID NO: 222 - RNA, 19 nt, Artificial Sequence
  Description of Artificial Sequence Synthetic oligonucleotide
gcuguacuuccaacaguua

SEQ ID NO: 223 - RNA, 19 nt, Artificial Sequence
  Description of Artificial Sequence Synthetic oligonucleotide
ccucgcaacucuuauaaau

SEQ ID NO: 224 - RNA, 19 nt, Artificial Sequence
  Description of Artificial Sequence Synthetic oligonucleotide
ccaagaggagacuuuaauu

SEQ ID NO: 225 - RNA, 25 nt, Artificial Sequence
  Description of Artificial Sequence Synthetic oligonucleotide
aaggcuugcacagggugaaagcuuu

SEQ ID NO: 226 - RNA, 25 nt, Artificial Sequence
  Description of Artificial Sequence Synthetic oligonucleotide
gagguauacuuaggcaaagagaaau

SEQ ID NO: 227 - RNA, 25 nt, Artificial Sequence
  Description of Artificial Sequence Synthetic oligonucleotide
aggcaaagagaaauuugacaguguu

SEQ ID NO: 228 - RNA, 25 nt, Artificial Sequence
  Description of Artificial Sequence Synthetic oligonucleotide
ucgcauccaccagaugaauucugaa

SEQ ID NO: 229 - RNA, 25 nt, Artificial Sequence
  Description of Artificial Sequence Synthetic oligonucleotide
acgagcaauaugaccaucuucugua

SEQ ID NO: 230 - RNA, 25 nt, Artificial Sequence
  Description of Artificial Sequence Synthetic oligonucleotide
ccaucuucuguauucuggaaacuga

SEQ ID NO: 231 - RNA, 25 nt, Artificial Sequence
  Description of Artificial Sequence Synthetic oligonucleotide
ccacauuccuuggauuacagcugua

SEQ ID NO: 232 - RNA, 25 nt, Artificial Sequence
  Description of Artificial Sequence Synthetic oligonucleotide
gauuacagcuguacuuccaacaguu

SEQ ID NO: 233 - RNA, 25 nt, Artificial Sequence
  Description of Artificial Sequence Synthetic oligonucleotide
ccauauaccugaaagaucugaugaa

SEQ ID NO: 234 - RNA, 25 nt, Artificial Sequence
  Description of Artificial Sequence Synthetic oligonucleotide
cguaugccaagaggagacuuuaauu

SEQ ID NO: 235 - RNA, 19 nt, Artificial Sequence
  Description of Artificial Sequence Synthetic oligonucleotide
ggcucuggaugacucugau

SEQ ID NO: 236 - RNA, 19 nt, Artificial Sequence
  Description of Artificial Sequence Synthetic oligonucleotide
gguggacuaggaaggcuuu

SEQ ID NO: 237 - RNA, 19 nt, Artificial Sequence
  Description of Artificial Sequence Synthetic oligonucleotide
gccaaucaagguacaagua

SEQ ID NO: 238 - RNA, 19 nt, Artificial Sequence
  Description of Artificial Sequence Synthetic oligonucleotide
ccaaucaagguacaaguaa

SEQ ID NO: 239 - RNA, 19 nt, Artificial Sequence
  Description of Artificial Sequence Synthetic oligonucleotide
gggcuucuuaagagagaau

SEQ ID NO: 240 - RNA, 19 nt, Artificial Sequence
  Description of Artificial Sequence Synthetic oligonucleotide
ggaaguggaggagaaucau

SEQ ID NO: 241 - RNA, 19 nt, Artificial Sequence
  Description of Artificial Sequence Synthetic oligonucleotide
ggagaaucaucucaggcaa

SEQ ID NO: 242 - RNA, 19 nt, Artificial Sequence
  Description of Artificial Sequence Synthetic oligonucleotide
ccuagucacagcuuuaaau

SEQ ID NO: 243 - RNA, 19 nt, Artificial Sequence
  Description of Artificial Sequence Synthetic oligonucleotide
gcaggaaucaagaucucaa

SEQ ID NO: 244 - RNA, 19 nt, Artificial Sequence
  Description of Artificial Sequence Synthetic oligonucleotide
ggaaagguguuucucucau

SEQ ID NO: 245 - RNA, 25 nt, Artificial Sequence
  Description of Artificial Sequence Synthetic oligonucleotide
gagguggacuaggaaggcuuucuga

SEQ ID NO: 246 - RNA, 25 nt, Artificial Sequence
  Description of Artificial Sequence Synthetic oligonucleotide
gcuuucugaagaaccugggucuguu

SEQ ID NO: 247 - RNA, 25 nt, Artificial Sequence
  Description of Artificial Sequence Synthetic oligonucleotide
ugggcuucuuaagagagaauaaguu

SEQ ID NO: 248 - RNA, 25 nt, Artificial Sequence
  Description of Artificial Sequence Synthetic oligonucleotide
cccucuuugugugaucacaugcaaa

SEQ ID NO: 249 - RNA, 25 nt, Artificial Sequence
  Description of Artificial Sequence Synthetic oligonucleotide
ccgacagcuccugaguuuauaucau

SEQ ID NO: 250 - RNA, 25 nt, Artificial Sequence
  Description of Artificial Sequence Synthetic oligonucleotide
agcuccugaguuuauaucaucucaa

SEQ ID NO: 251 - RNA, 25 nt, Artificial Sequence
  Description of Artificial Sequence Synthetic oligonucleotide
gcugugucuccuagucacagcuuua

SEQ ID NO: 252 - RNA, 25 nt, Artificial Sequence
  Description of Artificial Sequence Synthetic oligonucleotide
cagcccuguguaguuagagcaggaa

SEQ ID NO: 253 - RNA, 25 nt, Artificial Sequence
  Description of Artificial Sequence Synthetic oligonucleotide
gcuuagacguuaacuugaugcauca

SEQ ID NO: 254 - RNA, 25 nt, Artificial Sequence
  Description of Artificial Sequence Synthetic oligonucleotide
acguuaacuugaugcaucauuggaa

SEQ ID NO: 255 - RNA, 19 nt, Artificial Sequence
  Description of Artificial Sequence Synthetic oligonucleotide
ggcugggaucaugguagau

SEQ ID NO: 256 - RNA, 19 nt, Artificial Sequence
  Description of Artificial Sequence Synthetic oligonucleotide
gggaucaugguagauggaa

SEQ ID NO: 257 - RNA, 19 nt, Artificial Sequence
  Description of Artificial Sequence Synthetic oligonucleotide
cccacuccuugaaguauuu

SEQ ID NO: 258 - RNA, 19 nt, Artificial Sequence
  Description of Artificial Sequence Synthetic oligonucleotide
gcuucaucucugugggcua

SEQ ID NO: 259 - RNA, 19 nt, Artificial Sequence
  Description of Artificial Sequence Synthetic oligonucleotide
gguaugaacaguucgccua

SEQ ID NO: 260 - RNA, 19 nt, Artificial Sequence
  Description of Artificial Sequence Synthetic oligonucleotide
ggauuaucucacccugaau

SEQ ID NO: 261 - RNA, 19 nt, Artificial Sequence
  Description of Artificial Sequence Synthetic oligonucleotide
gccuaccuggaagacacau

SEQ ID NO: 262 - RNA, 19 nt, Artificial Sequence
  Description of Artificial Sequence Synthetic oligonucleotide
gcagagauacacgugccau

SEQ ID NO: 263 - RNA, 19 nt, Artificial Sequence
  Description of Artificial Sequence Synthetic oligonucleotide
ccuuggaucuguggucucu

SEQ ID NO: 264 - RNA, 19 nt, Artificial Sequence
  Description of Artificial Sequence Synthetic oligonucleotide
ccaccucugugucuaccau

SEQ ID NO: 265 - RNA, 25 nt, Artificial Sequence
  Description of Artificial Sequence Synthetic oligonucleotide
cgggcucccacuccuugaaguauuu

SEQ ID NO: 266 - RNA, 25 nt, Artificial Sequence
  Description of Artificial Sequence Synthetic oligonucleotide
cacuccuugaaguauuuccacacuu

SEQ ID NO: 267 - RNA, 25 nt, Artificial Sequence
  Description of Artificial Sequence Synthetic oligonucleotide
acggcaaggauuaucucacccugaa

SEQ ID NO: 268 - RNA, 25 nt, Artificial Sequence
  Description of Artificial Sequence Synthetic oligonucleotide
cggcaaggauuaucucacccugaau

SEQ ID NO: 269 - RNA, 25 nt, Artificial Sequence
  Description of Artificial Sequence Synthetic oligonucleotide
gauacacgugccaugugcagcauga

SEQ ID NO: 270 - RNA, 25 nt, Artificial Sequence
  Description of Artificial Sequence Synthetic oligonucleotide
uggagcugugguugcugcugugaua

SEQ ID NO: 271 - RNA, 25 nt, Artificial Sequence
  Description of Artificial Sequence Synthetic oligonucleotide
gcugugguugcugcugugauaugga

SEQ ID NO: 272 - RNA, 25 nt, Artificial Sequence
  Description of Artificial Sequence Synthetic oligonucleotide
uagcacaaugugaggagguagagaa

SEQ ID NO: 273 - RNA, 25 nt, Artificial Sequence
  Description of Artificial Sequence Synthetic oligonucleotide
gguagagaaacaguccaccucugug

SEQ ID NO: 274 - RNA, 25 nt, Artificial Sequence
  Description of Artificial Sequence Synthetic oligonucleotide
gagaaacaguccaccucugugucua

SEQ ID NO: 275 - RNA, 19 nt, Artificial Sequence
  Description of Artificial Sequence Synthetic oligonucleotide
ccuugaucaugugcccuaa

SEQ ID NO: 276 - RNA, 19 nt, Artificial Sequence
  Description of Artificial Sequence Synthetic oligonucleotide
gcucuuggcucucaacuua

SEQ ID NO: 277 - RNA, 19 nt, Artificial Sequence
  Description of Artificial Sequence Synthetic oligonucleotide
gcucucaacuuauucccuu

SEQ ID NO: 278 - RNA, 19 nt, Artificial Sequence
  Description of Artificial Sequence Synthetic oligonucleotide
gcuuguagcguacgacaau

SEQ ID NO: 279 - RNA, 19 nt, Artificial Sequence
  Description of Artificial Sequence Synthetic oligonucleotide
gcaaugaaucagugacauu

SEQ ID NO: 280 - RNA, 19 nt, Artificial Sequence
  Description of Artificial Sequence Synthetic oligonucleotide
gggaaacaccuuuguccaa

SEQ ID NO: 281 - RNA, 19 nt, Artificial Sequence
  Description of Artificial Sequence Synthetic oligonucleotide
gcuaguaacaguggccuuu

SEQ ID NO: 282 - RNA, 19 nt, Artificial Sequence
  Description of Artificial Sequence Synthetic oligonucleotide
gcaagcauuaccagcccua

SEQ ID NO: 283 - RNA, 19 nt, Artificial Sequence
  Description of Artificial Sequence Synthetic oligonucleotide
gcacaucucagucaagcaa

SEQ ID NO: 284 - RNA, 19 nt, Artificial Sequence
  Description of Artificial Sequence Synthetic oligonucleotide
ccacguaguuccuauuuaa

SEQ ID NO: 285 - RNA, 25 nt, Artificial Sequence
  Description of Artificial Sequence Synthetic oligonucleotide
ccuugugguuugagugccuugauca

SEQ ID NO: 286 - RNA, 25 nt, Artificial Sequence
  Description of Artificial Sequence Synthetic oligonucleotide
caggcugcucuuggcucucaacuua

SEQ ID NO: 287 - RNA, 25 nt, Artificial Sequence
  Description of Artificial Sequence Synthetic oligonucleotide
aggcugcucuuggcucucaacuuau

SEQ ID NO: 288 - RNA, 25 nt, Artificial Sequence
  Description of Artificial Sequence Synthetic oligonucleotide
gcggucaaccuuagcugcaaguauu

SEQ ID NO: 289 - RNA, 25 nt, Artificial Sequence
  Description of Artificial Sequence Synthetic oligonucleotide
cagugacauucuaccuccagaauuu

SEQ ID NO: 290 - RNA, 25 nt, Artificial Sequence
  Description of Artificial Sequence Synthetic oligonucleotide
gcaauggaaccauuauccaugugaa

SEQ ID NO: 291 - RNA, 25 nt, Artificial Sequence
  Description of Artificial Sequence Synthetic oligonucleotide
gggagggauaggaagacauauuuaa

SEQ ID NO: 292 - RNA, 25 nt, Artificial Sequence
  Description of Artificial Sequence Synthetic oligonucleotide
aaugagccacguaguuccuauuuaa

SEQ ID NO: 293 - RNA, 25 nt, Artificial Sequence
  Description of Artificial Sequence Synthetic oligonucleotide
ucccugucaugagacuucaguguua

SEQ ID NO: 294 - RNA, 25 nt, Artificial Sequence
  Description of Artificial Sequence Synthetic oligonucleotide
caugagacuucaguguuaauguuca

SEQ ID NO: 295 - RNA, 19 nt, Artificial Sequence
  Description of Artificial Sequence Synthetic oligonucleotide
gggaucaaagcuaucuaua

SEQ ID NO: 296 - RNA, 19 nt, Artificial Sequence
  Description of Artificial Sequence Synthetic oligonucleotide
ccuugauucuguguggguu

SEQ ID NO: 297 - RNA, 19 nt, Artificial Sequence
  Description of Artificial Sequence Synthetic oligonucleotide
gauucuguguggguucaaa

SEQ ID NO: 298 - RNA, 19 nt, Artificial Sequence
  Description of Artificial Sequence Synthetic oligonucleotide
ccauggcuugccuuggauu

SEQ ID NO: 299 - RNA, 19 nt, Artificial Sequence
  Description of Artificial Sequence Synthetic oligonucleotide
ccagcuuugugugugagua

SEQ ID NO: 300 - RNA, 19 nt, Artificial Sequence
  Description of Artificial Sequence Synthetic oligonucleotide
ucugcaagguggagcucau

SEQ ID NO: 301 - RNA, 19 nt, Artificial Sequence
  Description of Artificial Sequence Synthetic oligonucleotide
gccauacuaccugggcaua

SEQ ID NO: 302 - RNA, 19 nt, Artificial Sequence
  Description of Artificial Sequence Synthetic oligonucleotide
ggcaacggaacccagauuu

SEQ ID NO: 303 - RNA, 19 nt, Artificial Sequence
  Description of Artificial Sequence Synthetic oligonucleotide
gcaacggaacccagauuua

SEQ ID NO: 304 - RNA, 19 nt, Artificial Sequence
  Description of Artificial Sequence Synthetic oligonucleotide
ggaacccagauuuauguaa

SEQ ID NO: 305 - RNA, 25 nt, Artificial Sequence
  Description of Artificial Sequence Synthetic oligonucleotide
cauaucugggaucaaagcuaucuau

SEQ ID NO: 306 - RNA, 25 nt, Artificial Sequence
  Description of Artificial Sequence Synthetic oligonucleotide
cauaaagccauggcuugccuuggau

SEQ ID NO: 307 - RNA, 25 nt, Artificial Sequence
  Description of Artificial Sequence Synthetic oligonucleotide
cgccagcuuugugugugaguaugca

SEQ ID NO: 308 - RNA, 25 nt, Artificial Sequence
  Description of Artificial Sequence Synthetic oligonucleotide
gaagucugugcggcaaccuacauga

SEQ ID NO: 309 - RNA, 25 nt, Artificial Sequence
  Description of Artificial Sequence Synthetic oligonucleotide
ggaaugaguugaccuuccuagauga

SEQ ID NO: 310 - RNA, 25 nt, Artificial Sequence
  Description of Artificial Sequence Synthetic oligonucleotide
accuuccuagaugauuccaucugca

SEQ ID NO: 311 - RNA, 25 nt, Artificial Sequence
  Description of Artificial Sequence Synthetic oligonucleotide
cauaggcaacggaacccagauuuau

SEQ ID NO: 312 - RNA, 25 nt, Artificial Sequence
  Description of Artificial Sequence Synthetic oligonucleotide
caacggaacccagauuuauguaauu

SEQ ID NO: 313 - RNA, 25 nt, Artificial Sequence
  Description of Artificial Sequence Synthetic oligonucleotide
cggaacccagauuuauguaauugau

SEQ ID NO: 314 - RNA, 25 nt, Artificial Sequence
  Description of Artificial Sequence Synthetic oligonucleotide
ccucuggauccuugcagcaguuagu

SEQ ID NO: 315 - RNA, 19 nt, Artificial Sequence
  Description of Artificial Sequence Synthetic oligonucleotide
ccaaguguccauaccucaa

SEQ ID NO: 316 - RNA, 19 nt, Artificial Sequence
  Description of Artificial Sequence Synthetic oligonucleotide
ggucuuucucacuucuguu

SEQ ID NO: 317 - RNA, 19 nt, Artificial Sequence
  Description of Artificial Sequence Synthetic oligonucleotide
gcuguccuguggucacaau

SEQ ID NO: 318 - RNA, 19 nt, Artificial Sequence
  Description of Artificial Sequence Synthetic oligonucleotide
gggcacauacgaguguguu

SEQ ID NO: 319 - RNA, 19 nt, Artificial Sequence
  Description of Artificial Sequence Synthetic oligonucleotide
gcugacuucccuacaccua

SEQ ID NO: 320 - RNA, 19 nt, Artificial Sequence
  Description of Artificial Sequence Synthetic oligonucleotide
gcagcaaacuggauuucaa

SEQ ID NO: 321 - RNA, 19 nt, Artificial Sequence
  Description of Artificial Sequence Synthetic oligonucleotide
gcuuugcaggaagugucua

SEQ ID NO: 322 - RNA, 19 nt, Artificial Sequence
  Description of Artificial Sequence Synthetic oligonucleotide
gcugcuggaaguagaauuu

SEQ ID NO: 323 - RNA, 19 nt, Artificial Sequence
  Description of Artificial Sequence Synthetic oligonucleotide
ggaaguagaauuuguccaa

SEQ ID NO: 324 - RNA, 19 nt, Artificial Sequence
  Description of Artificial Sequence Synthetic oligonucleotide
ggucaacuucagagacuau

SEQ ID NO: 325 - RNA, 25 nt, Artificial Sequence
  Description of Artificial Sequence Synthetic oligonucleotide
acaguguguguagaaggcuuguuua

SEQ ID NO: 326 - RNA, 25 nt, Artificial Sequence
  Description of Artificial Sequence Synthetic oligonucleotide
ggaaugacuacuaagggaaaguauu

SEQ ID NO: 327 - RNA, 25 nt, Artificial Sequence
  Description of Artificial Sequence Synthetic oligonucleotide
cagcaacggugacauuaccuuuguu

SEQ ID NO: 328 - RNA, 25 nt, Artificial Sequence
  Description of Artificial Sequence Synthetic oligonucleotide
gagcgaaugguaaagcuaaacuuua

SEQ ID NO: 329 - RNA, 25 nt, Artificial Sequence
  Description of Artificial Sequence Synthetic oligonucleotide
ugccuguuuguugugugcagcauau

SEQ ID NO: 330 - RNA, 25 nt, Artificial Sequence
  Description of Artificial Sequence Synthetic oligonucleotide
uagcugccaugucggagcuucuaau

SEQ ID NO: 331 - RNA, 25 nt, Artificial Sequence
  Description of Artificial Sequence Synthetic oligonucleotide
ccuguuugacuuaguugcucguauu

SEQ ID NO: 332 - RNA, 25 nt, Artificial Sequence
  Description of Artificial Sequence Synthetic oligonucleotide
cccugaugcuuuaacuguuaccaua

SEQ ID NO: 333 - RNA, 25 nt, Artificial Sequence
  Description of Artificial Sequence Synthetic oligonucleotide
uggcacuagucaaaguaccagaaua

SEQ ID NO: 334 - RNA, 25 nt, Artificial Sequence
  Description of Artificial Sequence Synthetic oligonucleotide
ggguuuacaucaaccaccuccucaa

SEQ ID NO: 335 - DNA, 21 bp, Artificial Sequence
  Description of Artificial Sequence Synthetic oligonucleotide
ctggctcaacgacgaaagata

SEQ ID NO: 336 - DNA, 21 bp, Artificial Sequence
  Description of Artificial Sequence Synthetic oligonucleotide
cacggtaagcaccaagaagga

SEQ ID NO: 337 - DNA, 21 bp, Artificial Sequence
  Description of Artificial Sequence Synthetic oligonucleotide
aagggtggaactgttgcataa

SEQ ID NO: 338 - protein, 163 aa, Artificial Sequence
  Description of Artificial Sequence Synthetic polypeptide
HHHKHHHKHHHKHHHHHHKHHHKHHHKHHHKHHHHNHHHHKHHHKHHHKHHHKHHHHHHKHHHKHHHKHHHKHHHHNHHHHKHHHKHHHKHHHKHHHHHHKHHHKHHHKHHHKHHHHNHHHHKHHHKHHHKHHHKHHHHHHKHHHKHHHKHHHKHHHHNHHHH

SEQ ID NO: 339 - protein, 40 aa, Artificial Sequence
  Description of Artificial Sequence Synthetic peptide
HHHKHHHKHHHKHHHHHHKHHHKHHHKHHHKHHHHNHHHH

SEQ ID NO: 340 - protein, 17 aa, Artificial Sequence
  Description of Artificial Sequence Synthetic peptide
KHHHKHHHKHHHKHHHK

SEQ ID NO: 341 - protein, 7 aa, Artificial Sequence
  Description of Artificial Sequence Synthetic peptide
LPSPKRT

SEQ ID NO: 342 - protein, 7 aa, Artificial Sequence
  Description of Artificial Sequence Synthetic peptide
LPSPKKT

SEQ ID NO: 343 - protein, 7 aa, Artificial Sequence
  Description of Artificial Sequence Synthetic peptide
PTSVPKT

(19) United States
(12) Patent Application Publication
(10) Pub. No.: US 2010/0028848 A1
(43) Pub. Date: Feb. 4, 2010

(54) Compositions and Methods of Using siRNA to Knockdown Gene Expression and to Improve Solid Organ and Cell Transplantation

(75) Inventors: Marie Denise Parker, West Yorkshire (GB); Julian Roy Pratt, West Yorkshire (GB); Yijia Liu, Gaithersburg, MD (US); Yang Lu, Rockville, MD (US); Martin Woodle, Bethesda, MD (US); Yuefeng Xie, Germantown, MD (US)

Correspondence Address: ROPES & GRAY LLP, PATENT DOCKETING 39/361, 1211 AVENUE OF THE AMERICAS, NEW YORK, NY, 10036-8704, US

(73) Assignee: Intradigm Corporation, Palo Alto, CA (US)

(21) Appl. No.: 12/085,873

(22) PCT Filed: Nov. 30, 2006
(86) PCT No.: PCT/US06/45933

§ 371 Date: Sep. 10, 2009

Related U.S. Application Data
(60) Provisional application No. 60/741,157, filed on Nov. 30, 2005.

(51) Int. Cl. A01N 1/02; C07H 21/04

(52) U.S. Cl. 435/1.1; 536/23.1

(57) ABSTRACT

This invention describes compositions and methods using siRNA to target various genes expressed in cells of transplanted organs or tissues and/or genes expressed in the host to improve the success of the transplantation.

This application claims the benefit of U.S. provisional application No. 60/741,157, the entire disclosure of which is incorporated herein by reference.

### FIELD OF THE INVENTION

The present invention provides compositions and methods for the prevention of allograft rejection or xenograft rejection and ischemia/reperfusion injury in solid organ or tissue transplantation using siRNA-mediated down regulation of gene expression.

### BACKGROUND OF THE INVENTION

Solid organ transplantation is the only effective therapy for the treatment of end-stage organ failure (1, 2). Transplant programs around the world have become increasingly successful and such operations are becoming increasingly routine (3, 4). Despite the impressive results of one-year survival rates, organ transplantation still faces major problems. The immune system poses the most significant barrier to the long term survival of the transplanted organs. Without life long treatment with powerful immunosuppressive agents to keep the immune response at bay, organ grafts will invariably be rejected. However, current anti-rejection drugs reduce systemic immunity nonselectively and increase the risk of opportunistic infections and tumour development on the long term. Therefore, alternative strategies are being sought.

The advancement of molecular techniques over the past decade has improved our understanding of the signals necessary to elicit both an immune response and ischemia/reperfusion injury. Agents designed to target these novel signals provide hope that they will eventually allow for the long-term, drug-free acceptance of transplanted organs.

Transplantation immunology refers to an extensive sequence of events that occurs after an allograft or a xenograft is removed from a donor and then transplanted into a recipient. Tissue is damaged at both the graft and the transplantation sites. An inflammatory reaction follows immediately, as does activation of biochemical cascades. A series of specific and nonspecific cellular responses ensues as antigens are recognized. Eventually, the damage is controlled through tissue repair and reinforcement; if damage is nonpathologic, the graft survives.

Antigen-independent causes of tissue damage (i.e., ischemia, hypothermia, reperfusion injury) are the result of mechanical trauma as well as disruption of the blood supply as the graft is harvested.

In contrast, antigen-dependent causes of tissue damage involve immune-mediated damage. Macrophages release cytokines (e.g., tumour necrosis factor, interleukin-1), which heighten the intensity of inflammation by stimulating inflammatory endothelial responses; these endothelial changes help recruit large numbers of T cells to the transplantation site. Damaged tissues release proinflammatory mediators (e.g., Hageman factor [factor XII]) that trigger several biochemical cascades. The clotting cascade induces fibrin and several related fibrinopeptides, which promote local vascular permeability and attract neutrophils and macrophages. The kinin cascade principally produces bradykinin, which promotes vasodilation, smooth muscle contraction, and increased vascular permeability.

The formation of an antibody-antigen complex (i.e., immune complex) activates the classic pathway of the complement system. C1q triggers the activation process when it docks onto antibodies within the immune complexes via the classical pathway, whilst complement factor C3 can recognize damaged cell surfaces as acceptors for alternative pathway activation.

Activated complement causes damage through the deposition of the membrane attack complex (e.g., C5b, C6, C7, C8, C9) and cell-bound ligands, such as C4b and C3b, which activate leukocytes bearing complement receptors. In addition, production of bioactive anaphylatoxins C5a and C3a causes the influx and activation of inflammatory cells. These chemoattractants also initiate mast cell degranulation, which releases several mediators. Histamine and 5-hydroxytryptamine increase vascular permeability. Prostaglandin E2 promotes vasodilation and vascular permeability. Leukotrienes B4 and D2 promote leukocyte accumulation and vascular permeability. Another means by which complement is activated is through tissue ischemia and reperfusion, which exposes phospholipids and mitochondrial proteins. These by-products activate complement directly through binding C1q or mannose-binding lectin or factor C3b.

Currently, successful transplantation of allografts requires the systemic use of immunosuppressive drugs. These can cause serious morbidity due to toxicity and increased susceptibility to cancer and infections. Local production of immunosuppressive molecules limited to the graft site would reduce the need for conventional, generalized immunosuppressive therapies and thus educe fewer side effects. This is particularly salient in a disease like type 1 diabetes, which is not immediately life-threatening yet islet allografts can effect a cure. Anti-CD4 strategy may be even more effective when a combination of antibodies are used; similar strategies may also prevent xenograft rejection. Suppressing the host's immune responses also increases the risk of cancer. Attempts to suppress the immune response to avoid graft rejection and graft versus host disease (GVHD) weaken the ability of the body to combat infectious agents (e.g., bacteria, viruses, fungi, etc.).

RNA interference (RNAi) compounds, the intermediate short interfering RNA oligonucleotides (siRNAs), provide a unique strategy for using a combination of multiple siRNA duplexes to target multiple disease-causing genes in the same treatment, since all siRNA duplexes are chemically homogenous with the same source of origin and the same manufacturing process (5, 6, 7, 8). Such siRNA inhibitors are expected to have much better clinical efficiency with minimum toxicity and safety concerns. Genetic modification is a promising therapeutic strategy for organ transplantation. Based on the attractive technology of RNA interference for silencing a particular gene expression (9, 10), siRNA therapy may represent an attractive and powerful approach in preventing ischemia/reperfusion injury as well as organ rejection in transplant recipients.

### SUMMARY OF THE INVENTION

This invention provides targeting polynucleotides that target immunomodulatory or immunoeffector genes present in cells of an organ to be donated to a recipient. Targets for these polynucleotides can be derived from sequences of immunomodulatory and immunoeffector genes listed in Tables 1-15 (see below). For example, the targeting polynucleotide may target sequences in the C3, ICAM1, VCAM-1, IFN-γ, IL-1, IL-6, IL-8, TNF-α, CD80, CD86, MHC-II, MHC-I, CD28, CTLA-4, or PV-B19 genes. The targeting polynucleotides can comprise siRNA duplexes that target one or more of the sequences listed in Tables 1-15. The targeting polynucleotide may be a single-stranded linear polynucleotide, a double-stranded linear polynucleotide, or a hairpin polynucleotide.

This invention also provides a method of suppressing rejection of a transplanted organ by contacting the organ with a composition comprising the targeting polynucleotide of the invention before transplanting the organ into a recipient. The method can be effective in down-regulating or inhibiting the expression of a target immunomodulatory or immunoeffector gene in an organ or a cell of an organ during storage before transplantation. In one embodiment, the organ is perfused with a composition comprising a targeting polynucleotide of the invention. In another embodiment, the organ is bathed or submerged in the composition comprising a targeting polynucleotide of the invention. The composition can also be administered to an organ recipient. In some embodiments of the invention, the organ may be the recipient's own organ. The recipient of the said organ can be human. Organs, tissues, and cells contacted with the composition comprising a targeting polynucleotide of the invention include the kidney, liver, lung, pancreas, heart, small bowel, cornea, epithelial cells, vascular endothelium, vascular smooth muscle cells, myocardium and passenger leukocytes resident in the organ at the time of transplantation.

The composition comprising the targeting polynucleotide of the invention can also comprise a carrier, including, but not limited to, perfusion fluid, Hyper Osmolar Citrate solution, PolyTran polymer solution, TargeTran nanoparticle solution, or University of Wisconsin solution. The composition can also comprise small molecule drugs, monoclonal antibody drugs, and other immune modulators. In some embodiments the composition comprises a plurality of the targeting polynucleotide of the invention. A composition can contain a plurality of targeting polynucleotides of the invention that can target a plurality of gene sequences. In one embodiment, the targeting polynucleotides are a cocktail that targets the C3, TNF-α, and IL-8 gene sequences.

### BRIEF DESCRIPTION OF THE DRAWINGS

FIG. 1 is a bar graph that shows the relative expression of C3 mRNA in rat renal cells. The cells were stimulated with IL-1 and IL-6 to increase C3 expression. Three candidate C3 siRNA sequences (C3-1, C3-2, C3-3) or FITC-labelled scrambled siRNA were transfected into the cells at various concentrations. One set of cells was treated with Lipofectamine and no siRNA (+lipofectamine) while another set was stimulated to produce C3 and treated with neither Lipofectamine nor siRNA (−lipofectamine). C3 mRNA levels were measured in the cells by Real Time PCR 48 hours after transfection. The dotted line indicates unstimulated cell C3 expression. The experiment showed the feasibility and efficacy of gene knockdown by siRNA. The C3-3 siRNA was selected as the candidate to use in further experiments.

FIG. 2 is a bar graph showing the relative expression of C3 mRNA in rat renal cells stimulated with IL-1 and IL-6 to increase C3 expression. These cells were also transfected with various concentrations of the C3-3 candidate sequence. Real Time PCR for C3 mRNA expression after 48 hours of stimulation indicated that this siRNA sequence produced a reduction in C3 expression compared to stimulated cells treated with no siRNA. Measurements were normalized to unstimulated C3 mRNA expression in cells (dotted line).

FIG. 3 is a bar graph that shows the relative expression of C3 mRNA levels in transplanted rat kidneys. The kidneys were untreated or treated with nanoparticles containing various amounts of scrambled or C3 specific siRNA before transplantation. Each data point contains data from 4 separate kidneys, and each PCR reaction was performed in triplicate. C3 mRNA levels in these experimental conditions were compared to C3 mRNA levels in normal non-transplanted kidneys (NKC, normal kidney control) and transplanted kidneys untreated with siRNA (ISCH, ischaemic control). The figure demonstrates that C3 mRNA levels are lower in kidneys treated with C3 specific siRNA before transplantation as compared to C3 mRNA levels in normal non-transplanted kidneys and transplanted kidneys untreated with C3 specific siRNA. The C3 specific siRNA was packaged with various ratios of PolyTran, labelled in FIG. 1 as follows: C3, 10 μg C3 siRNA in PolyTran at 1:4.5; C3 naked, 10 μg C3 siRNA with no PolyTran; C3 3:1, 10 μg C3 siRNA in PolyTran at 1:3; C3 1.5:1, 10 μg C3 siRNA in PolyTran at 1:1.5. In order to test the requirement for siRNA specificity, two sets of kidneys were treated with scrambled siRNA before transplantation: FITC, 10 μg scrambled FITC-labeled siRNA; SCRAM CON, 10 μg scrambled non-labeled siRNA.

FIG. 4 is a set of two panels showing histological analysis of transplanted rat kidneys. The upper panel shows a non-treated kidney 48 hours after transplantation. The histopathology reveals widespread tubular attenuation and tubule dilation indicative of acute tubular necrosis (ATN). This particular pathology is linked to the initial non-function of transplanted tissue after transplantation. The lower panel depicts a kidney pre-treated with C3 siRNA (in 1:4.5 ratio with PolyTran) at 48 hours after transplantation. The histopathology of this kidney exhibits less ATN.

FIG. 5 shows two bar graphs presenting the results of an experiment serving to identify short peptides that can be used to target siRNA-comprising nanoparticles to specific organs. Phage display was used to identify candidate peptides that are concentrated in the transplanted kidney. The upper panel of FIG. 5 shows illustrative data for one experiment, with increasing concentrations of phage (in plaque forming units per gram of tissue) retrieved from the kidneys after three rounds of phage library injection, retrieval, and expansion. In a control experiment, streptavidin was used as a target for phage binding (R3vsStrep). The lower panel of FIG. 5 shows the number of phage retrieved after the third round of biopanning in the recipient's transplanted kidney (Tx kidney), normal kidney (N kidney), pancreas, heart, and lungs. The data shows selectivity in phage homing into the transplanted kidney compared to the numbers of phage retrieved from other organs.

### DETAILED DESCRIPTION OF THE INVENTION

As used herein, “oligonucleotides” and similar terms based on this relate to short polymers composed of naturally occurring nucleotides as well as to polymers composed of synthetic or modified nucleotides, as described in the immediately preceding paragraph. Oligonucleotides may be 10 or more nucleotides in length, or 15, or 16, or 17, or 18, or 19, or 20 or more nucleotides in length, or 21, or 22, or 23, or 24 or more nucleotides in length, or 25, or 26, or 27, or 28 or 29, or 30 or more nucleotides in length, 35 or more, 40 or more, 45 or more, up to about 50, nucleotides in length. An oligonucleotide that is an siRNA may have any number of nucleotides between 15 and 30 nucleotides. In many embodiments an siRNA may have any number of nucleotides between 21 and 25 nucleotides.

In many embodiments, an siRNA may have two blunt ends, or two sticky ends, or one blunt end with one sticky end, or one end with over hang. The over hang nucleotides can be ranged from one to four or more.

RNA interference (RNAi)

According to the invention, gene expression of immunomodulatory or immunoeffector gene targets is attenuated by RNA interference. Expression products of a immunomodulatory or immunoeffector gene are targeted by specific double stranded siRNA nucleotide sequences that are complementary to at least a segment of the immunomodulatory or immunoeffector gene target sequence that contains any number of nucleotides between 15 and 30, or in many cases, contains anywhere between 21 and 25 nucleotides, or more. The target may occur in the 5′ untranslated (UT) region, in a coding sequence, or in the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety.

According to the methods of the present invention, immunomodulatory or immunoeffector gene expression, and thereby ischemia/reperfusion injury or organ transplant rejection due to an adverse immunological reaction, is suppressed using siRNA. A targeting polynucleotide according to the invention includes an siRNA oligonucleotide. Such an siRNA can also be prepared by chemical synthesis of nucleotide sequences identical or similar to an intended sequence. See, e.g., Tuschl, Zamore, Lehmann, Bartel and Sharp (1999), Genes & Dev. 13: 3191-3197, incorporated herein by reference in its entirety. Alternatively, a targeting siRNA can be obtained using a targeting polynucleotide sequence, for example, by digesting an immunomodulatory or immunoeffector ribopolynucleotide sequence in a cell-free system, such as, but not limited to, a Drosophila extract, or by transcription of recombinant double stranded cRNA.

Efficient silencing is generally observed with siRNA duplexes composed of a 16-30 nt sense strand and a 16-30 nt antisense strand of the same length. In many embodiments each strand of an siRNA paired duplex has in addition a 2-nt overhang at the 3′ end. The sequence of the 2-nt 3′ overhang makes an additional small contribution to the specificity of siRNA target recognition. In one embodiment, the nucleotides in the 3′ overhang are ribonucleotides. In an alternative embodiment, the nucleotides in the 3′ overhang are deoxyribonucleotides. Use of 3′ deoxynucleotides provides enhanced intracellular stability.

A recombinant expression vector of the invention, when introduced within a cell, is processed to provide an RNA that comprises an siRNA sequence targeting an immunomodulatory or immunoeffector gene within the organ. Such a vector may be a DNA molecule cloned into an expression vector comprising operatively-linked regulatory sequences flanking the immunomodulatory or immunoeffector gene targeting sequence in a manner that allows for expression. From the vector, an RNA molecule that is antisense to the target RNA is transcribed by a first promoter (e.g., a promoter sequence 3′ of the cloned DNA) and an RNA molecule that is the sense strand for the RNA target is transcribed by a second promoter (e.g., a promoter sequence 5′ of the cloned DNA). The sense and antisense strands then hybridize in vivo to generate siRNA constructs targeting an immunomodulatory or immunoeffector gene sequence. Alternatively, two constructs can be utilized to create the sense and anti-sense strands of an siRNA construct. Further, cloned DNA can encode a transcript having secondary structure, wherein a single transcript has both the sense and complementary antisense sequences from the target gene or genes. In an example of this embodiment, a hairpin RNAi product is similar to all or a portion of the target gene. In another example, a hairpin RNAi product is an siRNA. The regulatory sequences flanking the immunomodulatory or immunoeffector gene sequence may be identical or may be different, such that their expression may be modulated independently, or in a temporal or spatial manner.

In certain embodiments, siRNAs are transcribed intracellularly by cloning the immunomodulatory or immunoeffector gene sequences into a vector containing, e.g., an RNA pol III transcription unit from the smaller nuclear RNA (snRNA) U6 or the human RNase P RNA H1. One example of a vector system is the GeneSuppressor™ RNA Interference kit (Imgenex Corp.). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50 nucleotide RNA stem loop transcript. The characteristics of RNAi and of factors affecting siRNA efficacy have been studied (See, e.g., Elbashir, Lendeckel and Tuschl (2001). Genes & Dev. 15: 188-200).

The targeting polynucleotide is generally 300 nucleotides in length or less, and includes a first nucleotide sequence that targets a gene sequence present in cells of the donated organ, or in passenger cells accompanying the donated organ once removed from the donor, and that is implicated in immunomodulatory or immunoeffector responses when a donated organ is introduced within a recipient subject. In the polynucleotide any T (thymidine) or any U (uridine) may optionally be substituted by the other. Additionally, in the polynucleotide the first nucleotide sequence consists of a) a sequence whose length is any number of nucleotides from 15 to 30, or more, or b) a complement of a sequence given in a). Such a polynucleotide may be termed a linear polynucleotide herein. A single stranded polynucleotide frequently is one strand of a double stranded siRNA.

In a related aspect, the polynucleotide described above further includes a second nucleotide sequence separated from the first nucleotide sequence by a loop sequence, such that the second nucleotide sequence.
a) has substantially the same length as the first nucleotide sequence, and
b) is substantially complementary to the first nucleotide sequence.

In this latter structure, termed a hairpin polynucleotide, the first nucleotide sequence hybridizes with the second nucleotide sequence to form a hairpin whose complementary sequences are linked by the loop sequence. A hairpin polynucleotide is digested intracellularly to form a double stranded siRNA.

In many embodiments the targets of the linear polynucleotide and of the hairpin polynucleotide are a gene sequence present in cells of the donated organ, or in passenger cells accompanying the donated organ, and the first nucleotide sequence is either.
a) a targeting sequence that targets a sequence chosen from the sequences given in Tables 1-15 appended hereto;
b) a targeting sequence longer than the sequence given in item a) wherein the targeting sequence targets a sequence chosen from Tables 1-15,
c) a fragment of a sequence given in a) or b) wherein the fragment consists of a sequence of contiguous bases at least 15 nucleotides in length and at most one base shorter than the chosen sequence,
d) a targeting sequence wherein up to 5 nucleotides differ from a sequence given in a)-c), or
e) a complement of any sequence given in a) to d).

In various embodiments of a linear polynucleotide or a hairpin polynucleotide the length of the first nucleotide sequence is any number of nucleotides from 21 to 25.

In many embodiments a linear polynucleotide or a hairpin polynucleotide consists of a targeting sequence that targets a sequence chosen from Tables 1-15, and optionally includes a dinucleotide overhang bound to the 3′ of the chosen sequence. In yet additional embodiments of a linear polynucleotide or a hairpin polynucleotide the dinucleotide sequence at the 3′ end of the first nucleotide sequence is TT, TU, UT, or UU and includes either ribonucleotides or deoxyribonucleotides or both. In various further embodiments a linear or hairpin polynucleotide may be a DNA, or it may be an RNA, or it may be composed of both deoxyribonucleotides and ribonucleotides.

Exemplary sequences of siRNA oligos specific to particular human genes are listed in Tables 1a to 15b below. The tables include both 21 mers with overhang and 25 mers with blunt ends for all the genes listed. The sequences of potential siRNA oligos specific to genes of other mammalian animals that are the transplantation donors should be designed in reference to the corresponding human genes but with the gene sequences of those animals in mind.

TABLE 1
siRNA targeted sequences in C3 gene:
C3 gene: Homo sapiens complement component 3 (C3),
Accession: NM_000064, Gene ID: 4557384, 25 siRNA
candidates were selected targeting the following
gene sequences:

Table 1a. 23 mer sequences (SEQ ID NOS 1-25):

| | | | | Thermo- |
|---|---|---|---|---|
| | | | | dynamic |
| # | Position | Sequence | GC% | Values |
| 1 | 1858-1880 | AAGGGCGTGTTCGTGCTGAATAA | 58 | −6.9 |
| | | | | (−13.5, |
| | | | | −6.6) |
| 2 | 2797-2819 | AAGGCTGCCGTCTACCATCATTT | 58 | −5.3 |
| | | | | (−12.1, |
| | | | | −6.8) |
| 3 | 3053-3075 | AACGGCTGAAGCACCTCATTGTG- | 58 | −4.9 |
| | | | | (−11.7, |
| | | | | −6.8) |
| 4 | 586-608 | AAGCAGGACTCCTTGTCTTCTCA | 53 | −4.6 |
| | | | | (−12.1, |
| | | | | −7.5) |
| 5 | 4163-4185 | AACCAGCACCGGAAACAGAAAAG | 53 | −4.6 |
| | | | | (−11.5, |
| | | | | −6.9) |
| 6 | 851-873 | AAGTGGAGGGAACTGCCTTTGTC | 58 | −4.5 |
| | | | | (−11.2, |
| | | | | −6.7) |
| 7 | 805-827 | AAGGGCCTGGAGGTCACCATCAC | 68 | −4.4 |
| | | | | (−14.4, |
| | | | | −10.0) |
| 8 | 4903-4925 | AAGCCCAACCTCAGCTACATCAT | 58 | −4.2 |
| | | | | (−13.2, |
| | | | | −9.0) |
| 9 | 3572-3594 | AAGCAGGAGACTTCCTTGAAGCC | 53 | −4.0 |
| | | | | (−12.1, |
| | | | | −8.1) |
| 10 | 1161-1183 | AATGCCCTTTGACCTCATGGTGT | 53 | −3.9 |
| | | | | (−12.7, |
| | | | | −8.8) |
| 11 | 4118-4140 | AAGATCAACTCACCTGTAATAAA | 37 | −3.8 |
| | | | | (−9.1, |
| | | | | −5.3) |
| 12 | 4663-4685 | AAGGCCTGTGAGCCAGGAGTGGA | 68 | −3.8 |
| | | | | (−13.2, |
| | | | | −9.4) |
| 13 | 2598-2620 | AATCCGAGCCGTTCTCTACAATT | 53 | −3.7 |
| | | | | (−10.9, |
| | | | | −7.2) |
| 14 | 925-947 | AAGCGCATTCCGATTGAGGATGG | 53 | −3.6 |
| | | | | (−12.5, |
| | | | | −8.9) |
| 15 | 2848-2870 | AAGGTCGTGCCGGAAGGAATCAG | 63 | −3.5 |
| | | | | (−11.4, |
| | | | | −7.9) |
| 16 | 2770-2792 | AAGACCGGCCTGCAGGAAGTGGA | 68 | −3.4 |
| | | | | (−11.4, |
| | | | | −8.0) |
| 17 | 4843-4865 | AAGCTGGAGGAGAAGAAACACTA | 53 | −3.4 |
| | | | | (−12.1, |
| | | | | −8.7) |
| 18 | 2097-2119 | AATGGACAAAGTCGGCAAGTACC | 47 | −3.4 |
| | | | | (−10.6, |
| | | | | −7.2) |
| 19 | 4549-4571 | AAGGAGGATGGAAAGCTGAACAA | 53 | −3.3 |
| | | | | (−12.1, |
| | | | | −8.8) |
| 20 | 4183-4205 | AAGAGGCCTCAGGATGCCAAGAA | 63 | −3.3 |
| | | | | (−12.3, |
| | | | | −9.0) |
| 21 | 337-359 | AACAGGGAGTTCAAGTCAGAAAA | 47 | −3.2 |
| | | | | (−11.3, |
| | | | | −8.1) |
| 22 | 1135-1157 | AAGACACCCAAGTACTTCAAACC | 42 | −3.2 |
| | | | | (−10.1, |
| | | | | −6.9) |
| 23 | 673-695 | AAGATCCGAGCCTACTATGAAAA | 47 | −3.2 |
| | | | | (−10.3, |
| | | | | −7.1) |
| 24 | 3890-3912 | AAGCCTTGGCTCAATACCAAAAG | 47 | −3.1 |
| | | | | (−10.9, |
| | | | | −7.8) |
| 25 | 4570-4592 | AAGCTCTGCCGTGATGAACTGTG | 58 | −3.1 |
| | | | | (−11.1, |
| | | | | −8.0) |

Table lb. 25 mer siRNA sense strand sequences
(SEQ ID NOS 26-35)

| | |
|---|---|
| 1: 2730 | CAAGUCCUCGUUGUCCGUUCCAUAU |
| 2: 2798 | AGGCUGCCGUCUACCAUCAUUUCAU |
| 3: 3504 | CAUCUCGCUGCAGGAGGCUAAAGAU |
| 4: 4113 | GGCCAAAGAUCAACUCACCUGUAAU |
| 5: 4199 | CCAAGAACACUAUGAUCCUUGAGAU |
| 6: 4272 | CAUAUCCAUGAUGACUGGCUUUGCU |
| 7: 4324 | GCCAAUGGUGUUGACAGAUACAUCU |
| 8: 4357 | GAGCUGGACAAAGCCUUCUCCGAUA |
| 9: 4672 | GAGCCAGGAGUGGACUAUGUGUACA |
| 10: 5012 | CCUUCACCGAGAGCAUGGUUGUCUU |

TABLE 2

siRNA targeted sequences in ICAM1 gene:
ICAM1 gene: Homo sapiens intercellular adhesion molecule 1
(CD54), human rhinovirus receptor (ICAM1), Accession:
NM_000201, Gene ID: 4557877, 19 siRNA candidates were
selected targeting the following gene sequences:

Tabl3 2a. 23 mer DNA sense strand sequences (SEQ ID NOS 36-54):

| # | Position Values | Sequence | GC % | Thermodynamic |
|---|---|---|---|---|
| 1 | 1567-1589 | AACCGCCAGCGGAAGATCAAGAA | 63 | −4.8 (−12.9, −8.1) |
| 2 | 280-302 | AACCGGAAGGTGTATGAACTGAG | 53 | −3.8 (−11.8, −8.0) |
| 3 | 641-663 | AAGGGCTGGAGCTGTTTGAGAAC | 58 | −3.7 (−13.2, −9.5) |
| 4 | 1291-1313 | AATTCCCAGCAGACTCCAATGTG | 53 | −3.6 (−10.4, −6.8) |
| 5 | 1533-1555 | AATGGGCACTGCAGGCCTCAGCA | 68 | −3.5 (−12.7, −9.2) |
| 6 | 286-308 | AAGGTGTATGAACTGAGCAATGT | 42 | −3.4 (−11.1, −7.7) |
| 7 | 1028-1050 | AAGGGACCGAGGTGACAGTGAAG | 63 | −2.9 (−12.3, −9.4) |
| 8 | 311-333 | AAGAAGATAGCCAACCAATGTGC | 42 | −2.4 (−8.9, −6.5) |
| 9 | 1210-1232 | AACCAGACCCGGGAGCTTCGTGT | 68 | −2.4 (−10.4, −8.0) |
| 10 | 1327-1349 | AACCCATTGCCCGAGCTCAAGTG | 63 | −2.2 (−10.3, −8.1) |
| 11 | 340-362 | AACTGCCCTGATGGGCAGTCAAC | 63 | −2.1 (−11.5, −9.4) |
| 12 | 1012-1034 | AAGCCAGAGGTCTCAGAAGGGAC | 63 | −2.0 (−12.1, −10.1 |
| 13 | 277-299 | AACAACCGGAAGGTGTATGAACT | 47 | −2.0 (−9.1, −7.1) |
| 14 | 874-896 | AAGGCCTCAGTCAGTGTGACCGC | 63 | −2.0 (−13.2, −11.2 |
| 15 | 323-345 | AACCAATGTGCTATTCAAACTGC | 37 | −1.7 (−8.0, −6.3) |
| 16 | 133-155 | AATGCCCAGACATCTGTGTCCCC | 58 | −1.5 (−12.7, −11.2 |
| 17 | 1048-1070 | AAGTGTGAGGCCCACCCTAGAGC | 63 | −1.5 (−9.9, −8.4) |
| 18 | 943-965 | AACCAGAGCCAGGAGACACTGCA | 63 | −1.3 (−10.4, −9.1) |
| 19 | 296-318 | AACTGAGCAATGTGCAAGAAGAT | 47 | −1.2 (−9.2, −8.0) |

Table 2b. 25 mer siRNA sense strand sequences (SEQ ID NOS 55-64):

| | |
|---|---|
| 1: 300 | GAGCAAUGUGCAAGAAGAUAGCCAA |
| 2: 316 | GAUAGCCAACCAAUGUGCUAUUCAA |
| 3: 345 | CCCAGAUGGGCAGUCAACAGCUAAA |
| 4: 1510 | ACUGUGGUAGCAGCCGCAGUCAUAA |
| 5: 1544 | CAGGCCUCAGCACGUACCUCUAUAA |
| 6: 1712 | CCACACUGAACAGAGUGGAAGACAU |
| 7: 1783 | GCAUUGUCCUCAGUCAGAUACAACA |
| 8: 1853 | CAUCUGAUCUGUAGUCACAUGACUA |
| 9: 1884 | GAGGAAGGAGCAAGACUCAAGACAU |
| 10: 1977 | GGACAUACAACUGGGAAAUACUGAA |

TABLE 3

siRNA targeted sequences in VCAM1 gene:
VCAM1 gene: Homo sapiens vascular cell adhesion molecule 1
(VCAM1), Transcript variant 2, mRNA. ACCESSION NM_080682.
GI: 18201908; transcript variant 1, mRNA. ACCESSION
NM_001078, GI: 18201907; Human vascular cell adhesion
molecule 1 mRNA, complete cds
gi|179885|gb|M30257.1|HUMCAM1V[179885], Human vascular cell
adhesion molecule 1 mRNA, complete cds,
gi|340193|gb|M60335.1|HUMVCAM1[340193], Human vascular cell
adhesion molecule-1 (VCAM1) gene, complete CDS,
gi|340195|gb|M73255.1|HUMVCAM1A[340195], Human mRNA for
vascular cell adhesion molecule 1 (VCAM-1),
gi|37648|emb|X53051.1|HSVCAM1[37648]25 siRNA candidates were
selected to target the following gene sequences:

Table 3a. 23 mer DNA sense strand sequences (SEQ ID NOS 65-89):

| # | Position | Sequence | GC % | Thermodynamic Values |
|---|---|---|---|---|
| 1 | 1858-1880 | AAGGGCGTGTTCGTGCTGAATAA | 58 | −6.9 (−13.5, −6.6) |
| 2 | 2797-2819 | AAGGCTGCCGTCTACCATCATTT | 58 | −5.3 (−21.1, −6.8) |
| 3 | 3053-3075 | AACGGCTGAAGCACCTCATTGTG | 58 | −4.9 (−11.7, −6.8) |
| 4 | 586-608 | AAGCAGGACTCCTTGTCTTCTCA | 53 | −4.6 (−12.1, −7.5) |
| 5 | 4163-4185 | AACCAGCACCGGAAACAGAAAAG | 53 | −4.6 (−11.5, −6.9) |
| 6 | 851-873 | AAGTGGAGGGAACTGCCTTTGTC | 58 | −4.5 (−11.2, −6.7) |
| 7 | 805-827 | AAGGGCCTGGAGGTCACCATCAC | 58 | −4.4 (−14.4, −10.0) |
| 8 | 4903-4925 | AAGCCCAACCTCAGCTACATCAT | 58 | −4.2 (−13.2, −9.0) |
| 9 | 3572-3594 | AAGCAGGAGACTTCCTTGAAGCC | 53 | −4.0 (−12.1, −8.1) |
| 10 | 1161-1183 | AATGCCCTTTGACCTCATGGTGT | 53 | −3.9 (−12.7, −8.8) |
| 11 | 4118-4140 | AAGATCAACTCACCTGTAATAAA | 37 | −3.8 (−9.1, −5.3) |
| 12 | 4663-4685 | AAGGCCTGTGAGCCAGGAGTGGA | 68 | −3.8 (−13.2, −9.4) |
| 13 | 2598-2620 | AATCCGAGCCGTTCTCTACAATT | 53 | −3.7 (−10.9, −7.2) |
| 14 | 925-947 | AAGCGCATTCCGATTGAGGATGG | 53 | −3.6 (−12.5, −8.9) |
| 15 | 2848-2870 | AAGGTCGTGCCGGAAGGAATCAG | 63 | −3.5 (−11.4, −7.9) |
| 16 | 2770-2792 | AAGACCGGCCTGCAGGAAGTGGA | 68 | −3.4 (−11.4, −8.0) |
| 17 | 4843-4865 | AAGCTGGAGGAGAAGAAACACTA | 53 | −3.4 (−12.1, −8.7) |
| 18 | 2097-2119 | AATGGACAAAGTCGGCAAGTACC | 47 | −3.4 (−10.6, −7.2) |
| 19 | 4549-4571 | AAGGAGGATGGAAAGCTGAACAA | 53 | −3.3 (−12.1, −8.8) |
| 20 | 4183-4205 | AAGAGGCCTCAGGATGCCAAGAA | 63 | −3.3 (−12.3, −9.0) |
| 21 | 337-359 | AACAGGGAGTTCAAGTCAGAAAA | 47 | −3.2 (−11.3, −8.1) |
| 22 | 1135-1157 | AAGACACCCAAGTACTTCAAACC | 42 | −3.2 (−10.1, −6.9) |
| 23 | 673-695 | AAGATCCGAGCCTACTATGAAAA | 47 | −3.2 (−10.3, −7.1) |
| 24 | 3890-3912 | AAGCCTTGGCTCAATACCAAAAG | 47 | −3.1 (−10.9, −7.8) |
| 25 | 4570-4592 | AAGCTCTGCCGTGATGAACTGTG | 58 | −3.1 (−11.1, −8.0) |

Table 3b. 25 mer siRNA sense strand sequences
(SEQ ID NOS 90-99):

| | |
|---|---|
| 1: 138 | CGUGAUCCUUGGAGCCUCAAAUAUA |
| 2: 212 | CAGAAUCUAGAUAUCUUGCUCAGAU |
| 3: 229 | GCUCAGAUUGGUGACUCCGUCUCAU |
| 4: 299 | GAACCCAGAUAGAUAGUCCACUGAA |
| 5: 439 | GGAAUCCAGGUGGAGAUCUACUCUU |
| 6: 645 | CAAGAGUUUGGAAGUAACCUUUACU |
| 7: 740 | UGCCCACAGUAAGGCAGGCUGUAAA |
| 8: 1046 | AAGCAUUCCCUAGAGAUCCAGAAAU |
| 9: 1687 | GAAGGAGACACUGUCAUCAUCUCUU |
| 10: 2106 | GCAAAUCCUUGAUACUGCUCAUCAU |

TABLE 4

siRNA sequences targeting human IFN-gamma
(Accession: NM_000619) (SEQ ID NOS 100-109):

Table 4a. 19 mer siRNA sense strand sequences:

| | |
|---|---|
| 1: 14 | UCAUCUGAAGAUCAGCUAU |
| 2: 56 | CCUUUGGACCUGAUCAGCU |
| 3: 477 | GCUGACUAAUUAUUCGGUA |
| 4: 510 | CCAACGCAAAGCAAUACAU |
| 5: 616 | GCAUCCCAGUAAUGGUUGU |
| 6: 912 | UCCCAUGGGUUGUGUGUUU |
| 7: 914 | CCAUGGGUUGUGUGUUUAU |
| 8: 1007 | GCAAUCUGAGCCAGUGCUU |
| 9: 1016 | GCCAGUGCUUUAAUGGCAU |
| 10: 1106 | GCUUCCAAAUAUUGUUGAC |

Table 4b. 25 mer siRNA sense strand sequences
(SEQ ID NOS 110-119):

| | |
|---|---|
| 1: 12 | GAUCAUCUGAAGAUCAGCUAUUAGA |
| 2: 47 | CAGUUAAGUCCUUUGGACCUGAUCA |
| 3: 494 | UAACUGACUUGAAUGUCCAACGCAA |
| 4: 604 | CGAGGUCGAAGAGCAUCCCAGUAAU |
| 5: 622 | CAGUAAUGGUUGUCCUGCCUGCAAU |
| 6: 626 | AAUGGUUGUCCUGCCUGCAAUAUUU |
| 7: 849 | GCAAGGCUAUGUGAUUACAAGGCUU |
| 8: 907 | CAAGAUCCCAUGGGUUGUGUGUUUA |
| 9: 918 | GGGUUGUGUGUUUAUUUCACUUGAU |
| 10: 1004 | CCUGCAAUCUGAGCCAGUGCUUUAA |

TABLE 5

siRNA sequences targeting human IL-1
(Accession: NM_033292):

Table 5a. 19 mer siRNA sense strand sequences
(SEQ ID NOS 120-129):

| | |
|---|---|
| 1: 767 | GCAAGUCCCAGAUAUACUA |
| 2: 826 | GCCCAAGUUUGAAGGACAA |
| 3: 827 | CCCAAGUUUGAAGGACAAA |
| 4: 885 | CCUGGUGUGGUGUGGUUUA |
| 5: 909 | UCAGUAGGAGUUUCUGGAA |
| 6: 915 | GGAGUUUCUGGAAACCUAU |
| 7: 924 | GGAAACCAUACUUUACCAA |
| 8: 1180 | CCACUGAAAGAGUGACUUU |
| 9: 1270 | GAAGAGAUCCUUCUGUAAA |
| 10: 1296 | GGAAUUAUGUCUGCUGAAU |

Table 5b. 25 mer siRNA sense strand sequences
(SEQ ID NOS 130-139):

| | |
|---|---|
| 1: 769 | AAGUCCCAGAUAUACUACAACUCAA |
| 2: 826 | GCCCAAGUUUGAAGGACAAACCGAA |
| 3: 881 | CAGCCCUGGUGUGGUGUGGUUUAAA |
| 4: 884 | CCCUGGUGUGGUGUGGUUUAAAGAU |
| 5: 887 | UGGUGUGGUGUGGUUUAAAGAUUCA |
| 6: 909 | UCAGUAGGAGUUUCUGGAAACCUAU |
| 7: 913 | UAGGAGUUUCUGGAAACCUAUCUUU |
| 8: 914 | AGGAGUUUCUGGAAACCUAUCUUUA |
| 9: 1176 | CCCACCACUGAAAGAGUGACUUUGA |
| 10: 1178 | CACCACUGAAAGAGUGACUUUGACA |

TABLE 6

siRNA sequences targeting human IL-6
(Accession: NM_000600):

Table 6a. 19 mer siRNA sense strand sequences
(SEQ ID NOS 140-149)

| | |
|---|---|
| 1: 250 | GCAUCUCAGCCCUGAGAAA |
| 2: 258 | GCCCUGAGAAAGGAGACAU |
| 3: 360 | GGAUGCUUCCAAUCUGGAU |
| 4: 364 | GCUUCCAAUCUGGAUUCAA |
| 5: 375 | GGAUUCAAUGAGGAGACUU |
| 6: 620 | GCAGGACAUGACAACUCAU |
| 7: 706 | GGCACCUCAGAUUGUUGUU |
| 8: 710 | CCUCAGAUUGUUGUUGUUA |
| 9: 768 | GCACAGAACUUAUGUUGUU |
| 10: 949 | GGAAAGUGGCUAUGCAGUU |

Table 2b. 25 mer siRNA sense strand sequences
(SEQ ID NOS 150-159)

| | |
|---|---|
| 1: 256 | CAGCCCUGAGAAAGGAGACAUGUAA |
| 2: 359 | UGGAUGCUUCCAAUCUGGAUUCAAU |
| 3: 429 | GAGGUAUACCUAGAGUACCUCCAGA |
| 4: 446 | CCUCCAGAACAGAUUUGAGAGUAGU |
| 5: 631 | CAACUCAUCUCAUUCUGCGCAGCUU |
| 6: 705 | GGGCACCUCAGAUUGUUGUUGUUAA |
| 7: 762 | CACUGGGCACAGAACUUAUGUUGUU |
| 8: 767 | GGCACAGAACUUAUGUUGUUCUCUA |
| 9: 768 | GCACAGAACUUAUGUUGUUCUCUAU |
| 10: 1002 | UGGAAAGUGUAGGCUUACCUCAAAU |

TABLE 7

siRNA sequences targeting human IL-8
(Accession: NM_000584):

Table 7a. 19 mer siRNA sense strand sequences
(SEQ ID NOS 160-168)

| | |
|---|---|
| 1: 1342 | ACUCCCAGUCUUGUCAUUG |
| 2: 1345 | CCCAGUCUUGUCAUUGCCA |
| 3: 1346 | CCAGUCUUGUCAUUGCCAG |
| 4: 1364 | GCUGUGUUGGUAGUGCUGU |
| 5: 1372 | GGUAGUGCUGUGUUGAAUU |
| 6: 1373 | GUAGUGCUGUGUUGAAUUA |
| 7: 1378 | GCUGUGUUGAAUUACGGAA |
| 8: 1379 | CUGUGUUGAAUUACGGAAU |
| 9: 1427 | ACUCCACAGUCAAUAUUAG |

Table 7a. 25 mer siRNA sense strand sequences
(SEQ ID NOS 169-174)

| | |
|---|---|
| 1: 1364 | GCUGUGUUGGUAGUGCUGUGUUGAA |
| 2: 1366 | UGUGUUGGUAGUGCUGUGUUGAAUU |
| 3: 1372 | GGUAGUGCUGUGUUGAAUUACGGAA |
| 4: 1374 | UAGUGCUGUGUUGAAUUACGGAAUA |
| 5: 1375 | AGUGCUGUGUUGAAUUACGGAAUAA |
| 6: 1378 | GCUGUGUUGAAUUACGGAAUAAUGA |

TABLE 8
siRNA sequences targeting human TNF-α
(Accession: NM_004862):
Table 8a. 19 mer siRNA sense strand sequences
(SEQ ID NOS 175-184)

| | |
|---|---|
| 1: 163 | GGACACCAUGAGCACUGAA |
| 2: 168 | CCAUGAGCACUGAAAGCAU |
| 3: 430 | GCCUGUAGCCCAUGUUGUA |
| 4: 516 | GCGUGGAGCUGAGAGAUAA |
| 5: 811 | GCCCGACUAUCUCGACUUU |
| 6: 993 | CCCAAGCUUAGAACUUUAA |
| 7: 1072 | GCUGGCAACCACUAAGAAU |
| 8: 1076 | GCAACCACUAAGAAUUCAA |
| 9: 1301 | GCCAGCUCCCUCUAUUUAU |
| 10: 1305 | GCUCCCUCUAUUUAUGUUU |

Table 8b. 25 mer siRNA sense strand sequences
(SEQ ID NOS 185-194)

| | |
|---|---|
| 1: 906 | UGGAGUCGUGCAUAGGACUUGCAAA |
| 2: 1002 | GAUCAUUGCCCUAUCCGAAUAUCUU |
| 3: 1010 | CCCUAUCCGAAUAUCUUCCUGUGAU |
| 4: 1146 | GAACCAGCCUUUAGUGCCUACCAUU |
| 5: 1150 | CAGCCUUUAGUGCCUACCAUUAUCU |
| 6: 1153 | CCUUUAGUGCCUACCAUUAUCUUAU |
| 7: 1199 | GACAAAGAUCUUGCCUUACAGACUU |
| 8: 1241 | GAUUCUGUAACUGCAGACUUCAUUA |
| 9: 1244 | UCUGUAACUGCAGACUUCAUUAGCA |
| 10: 1254 | CAGACUUCAUUAGCACACAGAUUCA |

TABLE 9
siRNA sequences targeting human CD80
(Accession: NM_005191):
Table 9a. 19 mer siRNA sense strand sequences
(SEQ ID NOS 195-204)

| | |
|---|---|
| 1: 398 | CCAAGUGUCCAUACCUCAA |
| 2: 442 | GGUCUUUCUCACUUCUGUU |
| 3: 504 | GCUGUCCUGUGGUCACAAU |
| 4: 696 | GGGCACAUACGAGUGUGUU |
| 5: 781 | GCUGACUUCCCUACACCUA |
| 6: 965 | GCAGCAAACUGGAUUUCAA |
| 7: 1378 | GCUUUGCAGGAAGUGUCUA |
| 8: 1652 | GCUGCUGGAAGUAGAAUUU |
| 9: 1658 | GGAAGUAGAAUUUGUCCAA |
| 10: 1682 | GGUCAACUUCAGAGACUAU |

Table 9b. 25 mer siRNA sense strand sequences
(SEQ ID NOS 205-214)

| | |
|---|---|
| 1: 535 | GAGCUGGCACAAACUCGCAUCUACU |
| 2: 599 | GGGACAUGAAUAUAUGGCCCGAGUA |
| 3: 631 | CGGACCAUCUUUGAUAUCACUAAUA |
| 4: 698 | GCACAUACGAGUGUGUUGUUCUGAA |
| 5: 898 | GGAGAAGAAUUAAAUGCCAUCAACA |
| 6: 1205 | GAAGGGAAAGUGUACGCCCUGUAUA |
| 7: 1275 | CCUCCAUUUGCAAUUGACCUCUUCU |
| 8: 1302 | GAACUUCCUCAGAUGGACAAGAUUA |
| 9: 1565 | CAGAUUUCCUAACUCUGGUGCUCUU |
| 10: 1766 | AGGAAGUAUGGCAUGAACAUCUUUA |

TABLE 10
siRNA sequences targeting human CD86
(Accession: NM_175862):
Table 10a. 19 mer siRNA sense strand sequence
(SEQ ID NOS 215-224)

| | |
|---|---|
| 1: 36 | GCUGCUGUAACAGGGACUA |
| 2: 130 | GCACUAUGGGACUGAGUAA |
| 3: 189 | CCUCUGAAGAUUCAAGCUU |
| 4: 398 | CCUGAGACUUCACAAUCUU |
| 5: 425 | GGACAAGGGCUUGUAUCAA |
| 6: 466 | CCACAGGAAUGAUUCGCAU |
| 7: 586 | GCUCAUCUAUACACGGUUA |
| 8: 867 | GCUGUACUUCCAACAGUUA |
| 9: 942 | CCUCGCAACUCUUAUAAAU |
| 10: 1284 | CCAAGAGGAGACUUUAAUU |

Table 10b. 25 mer siRNA sense strand sequence
(SEQ ID NOS 225-234)

| | |
|---|---|
| 1: 3 | AAGGCUUGCACAGGGUGAAAGCUUU |
| 2: 315 | GAGGUAUACUUAGGCAAAGAGAAAU |
| 3: 326 | AGGCAAAGAGAAAUUUGACAGUGUU |
| 4: 479 | UCGCAUCCACCAGAUGAAUUCUGAA |
| 5: 747 | ACGAGCAAUAUGACCAUCUUCUGUA |
| 6: 760 | CCAUCUUCUGUAUUCUGGAAACUGA |
| 7: 848 | CCACAUUCCUUGGAUUACAGCUGUA |
| 8: 860 | GAUUACAGCUGUACUUCCAACAGUU |
| 9: 1019 | CCAUAUACCUGAAAGAUCUGAUGAA |
| 10: 1278 | CGUAUGCCAAGAGGAGACUUUAAUU |

TABLE 11
siRNA sequences targeting human MHC-II
(Accession: NM_002119):
Table 11a. 19 mer siRNA sense strand sequences
(SEQ ID NOS 235-244)

| | |
|---|---|
| 1: 2474 | GGCUCUGGAUGACUCUGAU |
| 2: 2593 | GGUGGACUAGGAAGGCUUU |
| 3: 2641 | GCCAAUCAAGGUACAAGUA |
| 4: 2642 | CCAAUCAAGGUACAAGUAA |
| 5: 2740 | GGGCUUCUUAAGAGAGAAU |
| 6: 2790 | GGAAGUGGAGGAGAAUCAU |
| 7: 2799 | GGAGAAUCAUCUCAGGCAA |
| 8: 3149 | CCUAGUCACAGCUUUAAAU |
| 9: 3233 | GCAGGAAUCAAGAUCUCAA |
| 10: 3416 | GGAAAGGUGUUUCUCUCAU |

Table 11b. 25 mer siRNA sense strand sequences
(SEQ ID NOS 245-254)

| | |
|---|---|
| 1: 2591 | GAGGUGGACUAGGAAGGCUUUCUGA |
| 2: 2607 | GCUUUCUGAAGAACCUGGGUCUGUU |
| 3: 2739 | UGGGCUUCUUAAGAGAGAAUAAGUU |
| 4: 2843 | CCCUCUUUGUGUGAUCACAUGCAAA |
| 5: 3092 | CCGACAGCUCCUGAGUUUAUAUCAU |
| 6: 3097 | AGCUCCUGAGUUUAUAUCAUCUCAA |
| 7: 3140 | GCUGUGUCUCCUAGUCACAGCUUUA |
| 8: 3215 | CAGCCCUGUGUAGUUAGAGCAGGAA |
| 9: 3389 | GCUUAGACGUUAACUUGAUGCAUCA |
| 10: 3395 | ACGUUAACUUGAUGCAUCAUUGGAA |

TABLE 12
siRNA sequences targeting human MHC-I
(Accession: NM_005516)
Table 12a. 19 mer siRNA sense strand sequences
(SEQ ID NOS 255-264):

| | |
|---|---|
| 1: 29 | GGCUGGGAUCAUGGUAGAU |
| 2: 33 | GGGAUCAUGGUAGAUGGAA |
| 3: 106 | CCCACUCCUUGAAGUAUUU |
| 4: 163 | GCUUCAUCUCUGUGGGCUA |
| 5: 436 | GGUAUGAACAGUUCGCCUA |
| 6: 464 | GGAUUAUCUCACCCUGAAU |
| 7: 573 | GCCUACCUGGAAGACACAU |
| 8: 863 | GCAGAGAUACACGUGCCAU |
| 9: 980 | CCUUGGAUCUGUGGUCUCU |
| 10: 1296 | CCACCUCUGUGUCUACCAU |

Table 12b. 25 mer siRNA sense strand sequences
(SEQ ID NOS 265-274):

| | |
|---|---|
| 1: 100 | CGGGCUCCCACUCCUUGAAGUAUUU |
| 2: 108 | CACUCCUUGAAGUAUUUCCACACUU |
| 3: 457 | ACGGCAAGGAUUAUCUCACCCUGAA |
| 4: 458 | CGGCAAGGAUUAUCUCACCCUGAAU |
| 5: 868 | GAUACACGUGCCAUGUGCAGCAUGA |
| 6: 998 | UGGAGCUGUGGUUGCUGCUGUGAUA |
| 7: 1002 | GCUGUGGUUGCUGCUGUGAUAUGGA |
| 8: 1266 | UAGCACAAUGUGAGGAGGUAGAGAA |
| 9: 1282 | GGUAGAGAAACAGUCCACCUCUGUG |
| 10: 1286 | GAGAAACAGUCCACCUCUGUGUCUA |

TABLE 13
siRNA sequences targeting human CD28
(Accession: NM_006139):
Table 13a. 19 mer siRNA sense strand sequences
(SEQ ID NOS 275-284)

| | |
|---|---|
| 1: 69 | CCUUGAUCAUGUGCCCUAA |
| 2: 234 | GCUCUUGGCUCUCAACUUA |
| 3: 241 | GCUCUCAACUUAUUCCCUU |
| 4: 306 | GCUUGUAGCGUACGACAAU |
| 5: 494 | GCAAUGAAUCAGUGACAUU |
| 6: 631 | GGGAAACACCUUUGUCCAA |
| 7: 726 | GCUAGUAACAGUGGCCUUU |
| 8: 830 | GCAAGCAUUACCAGCCCUA |
| 9: 1216 | GCACAUCUCAGUCAAGCAA |
| 10: 1413 | CCACGUAGUUCCUAUUUAA |

Table 13b. 25 mer siRNA sense strand sequences
(SEQ ID NOS 285-294)

| | |
|---|---|
| 1: 53 | CCUUGUGGUUUGAGUGCCUUGAUCA |
| 2: 228 | CAGGCUGCUCUUGGCUCUCAACUUA |
| 3: 229 | AGGCUGCUCUUGGCUCUCAACUUAU |
| 4: 325 | GCGGUCAACCUUAGCUGCAAGUAUU |
| 5: 503 | CAGUGACAUUCUACCUCCAGAAUUU |
| 6: 605 | GCAAUGGAACCAUUAUCCAUGUGAA |
| 7: 1351 | GGGAGGGAUAGGAAGACAUAUUUAA |
| 8: 1407 | AAUGAGCCACGUAGUUCCUAUUUAA |
| 9: 1577 | UCCCUGUCAUGAGACUUCAGUGUUA |
| 10: 1584 | CAUGAGACUUCAGUGUUAAUGUUCA |

TABLE 14
siRNA sequences targeting human CTLA4
(Accession: AF414120):
Table 14a. 19 mer siRNA sense strand sequences
(SEQ ID NOS 295-304)

| | |
|---|---|
| 1: 33 | GGGAUCAAAGCUAUCUAUA |
| 2: 58 | CCUUGAUUCUGUGUGGGUU |
| 3: 62 | GAUUCUGUGUGGGUUCAAA |
| 4: 154 | CCAUGGCUUGCCUUGGAUU |
| 5: 316 | CCAGCUUUGUGUGUGAGUA |
| 6: 538 | UCUGCAAGGUGGAGCUCAU |
| 7: 566 | GCCAUACUACCUGGGCAUA |
| 8: 585 | GGCAACGGAACCCAGAUUU |
| 9: 586 | GCAACGGAACCCAGAUUUA |
| 10: 591 | GGAACCCAGAUUUAUGUAA |

Table 14b. 25 mer siRNA sense strand sequences
(SEQ ID NOS 305-314)

| | |
|---|---|
| 1: 26 | CAUAUCUGGGAUCAAAGCUAUCUAU |
| 2: 147 | CAUAAAGCCAUGGCUUGCCUUGGAU |
| 3: 314 | CGCCAGCUUUGUGUGUGAGUAUGCA |
| 4: 402 | GAAGUCUGUGCGGCAACCUACAUGA |
| 5: 430 | GGAAUGAGUUGACCUUCCUAGAUGA |
| 6: 441 | ACCUUCCUAGAUGAUUCCAUCUGCA |
| 7: 581 | CAUAGGCAACGGAACCCAGAUUUAU |
| 8: 587 | CAACGGAACCCAGAUUUAUGUAAUU |
| 9: 590 | CGGAACCCAGAUUUAUGUAAUUGAU |
| 10: 644 | CCUCUGGAUCCUUGCAGCAGUUAGU |

TABLE 15
siRNA sequences targeting human parvovirus B19
(Accession: AY903437):
Table 15a. 19 mer siRNA sense strand sequences
(SEQ ID NOS 315-324)

| | |
|---|---|
| 1: 398 | CCAAGUGUCCAUACCUCAA |
| 2: 442 | GGUCUUUCUCACUUCUGUU |
| 3: 504 | GCUGUCCUGUGGUCACAAU |
| 4: 696 | GGGCACAUACGAGUGUGUU |
| 5: 781 | GCUGACUUCCCUACACCUA |
| 6: 965 | GCAGCAAACUGGAUUUCAA |
| 7: 1378 | GCUUUGCAGGAAGUGUCUA |
| 8: 1652 | GCUGCUGGAAGUAGAAUUU |
| 9: 1658 | GGAAGUAGAAUUUGUCCAA |
| 10: 1682 | GGUCAACUUCAGAGACUAU |

Table 15b. 25 mer siRNA sense strand sequences
(SEQ ID NOS 325-334)

| | |
|---|---|
| 1: 729 | ACAGUGUGUGUAGAAGGCUUGUUUA |
| 2: 807 | GGAAUGACUACUAAGGGAAAGUAUU |
| 3: 1679 | CAGCAACGGUGACAUUACCUUUGUU |
| 4: 1749 | GAGCGAAUGGUAAAGCUAAACUUUA |
| 5: 2230 | UGCCUGUUUGUUGUGUGCAGCAUAU |
| 6: 2360 | UAGCUGCCAUGUCGGAGCUUCUAAU |
| 7: 2622 | CCUGUUUGACUUAGUUGCUCGUAUU |
| 8: 3474 | CCCUGAUGCUUUAACUGUUACCAUA |
| 9: 4083 | UGGCACUAGUCAAAGUACCAGAAUA |
| 10: 4470 | GGGUUUACAUCAACCACCUCCUCAA |

In one embodiment, siRNA duplexes of 25 basepair with blunt ends exhibit more potent gene knockdown efficacy than 19 basepair with overhang at both 3′ ends, both in vitro and in vivo.

In an additional aspect the invention provides a double stranded polynucleotide that includes a first linear polynucleotide strand described above and a second polynucleotide strand that is complementary to at least the first nucleotide sequence of the first strand and is hybridized thereto to form a double stranded siRNA composition.

### Formulations

A variety of carriers serve to prepare formulations or pharmaceutical compositions containing siRNAs. In several embodiments the siRNA polynucleotides of the invention are delivered into cells in culture or into cells of an organ awaiting transplantation by liposome-mediated transfection, for example by using commercially available reagents or techniques, e.g., Oligofectamine™, LipofectAmine™ reagent, LipofectAmine 2000™ (Invitrogen), as well as by electroporation, and similar techniques.

The pharmaceutical compositions containing the siRNAs include additional components that protect the stability of siRNA, prolong siRNA lifetime, potentiate siRNA function, or target siRNA to specific tissues/cells. These include a variety of biodegradable polymers, cationic polymers (such as polyethyleneimine), cationic copolypeptides such as histidine-lysine (HK) polypeptides see, for example, PCT publications WO 01/47496 to Mixson et al., WO 02/096941 to Biomerieux, and WO 99/42091 to Massachusetts Institute of Technology), PEGylated cationic polypeptides, and ligand-incorporated polymers, etc. positively charged polypeptides, PolyTran solutions (saline or aqueous solution of HK polymers and polysaccharides such as natural polysaccharides, also known as scleroglucan), TargeTran (a saline or aqueous suspension of nano-particle composed of conjugated RGD-PEG-PEI polymers including a targeting ligand), surfactants (Infasurf; Forest Laboratories, Inc.; ONY Inc.), and cationic polymers (such as polyethyleneimine). Infasurf® (calfactant) is a natural lung surfactant isolated from calf lung for use in intratracheal instillation; it contains phospholipids, neutral lipids, and hydrophobic surfactant-associated proteins B and C.

The polymers can either be uni-dimensional or multi-dimensional, and also could be microparticles or nanoparticles with diameters less than 20 microns, between 20 and 100 microns, or above 100 micron. The said polymers could carry ligand molecules specific for receptors or molecules of special tissues or cells, thus be used for targeted delivery of siRNAs. The siRNA polynucleotides are also delivered by cationic liposome based carriers, such as DOTAP, DOTAP/Cholesterol (Qbiogene, Inc.) and other types of lipid aqueous solutions. In addition, low percentage (5-10%) glucose aqueous solution, and Infasurf are effective carriers for airway delivery of siRNA (Li B. J. et al, 2005, Nature Medicine, 11, 944-951).

In addition, a carrier may include Hyper Osmolar Citrate solution (560 mOsm/kg solution of meglumine hydrochloride, 560 mOsm/kg meglumine ioxaglate, and 600 mOsm/kg sodium ioxaglate, and so forth). University of Wisconsin solution has the potential to enhance and extend heart, kidney, lung and liver preservation. University of Wisconsin solution is widely accepted for the cold storage and transport of human donor pancreata destined for islet isolation.

The composition may further comprise a polymeric carrier. The polymeric carrier may comprise a cationic polymer that binds to the RNA molecule. The cationic polymer may be an amino acid copolymer, comprising, for example, histidine and lysine residues. The polymer may comprise a branched polymer.

The composition may comprise a targeted synthetic vector. The synthetic vector may comprise a cationic polymer, a hydrophilic polymer, and a targeting ligand. The polymer may comprise a polyethyleneimine, the hydrophilic polymer may comprise a polyethylene glycol or a polyacetal, and the targeting ligand may comprise a peptide comprising an RGD sequence.

The siRNA/carrier may be formulated in either the storage solution or the perfusion medium in a non-specific manner, or via the systemic circulation in a targeted delivery system.

### Improving Solid Organ And Cell Transplantation

The present invention provides methods for prevention of allograft rejection and ischemia/reperfusion injury in solid organ transplantation by silencing or down-regulation of a target gene expression by introducing RNA interference (siRNA). In a method of the present invention, siRNA is applied to an organ intended for transplantation in the form of an organ-storage solution, i.e., after removal from the donor and while it is being transported to the recipient. The donor or recipient of the transplanted organ, tissues, and/or cells can be a mammal, including, but not limited to, human, non-human mammal, non-human primate, rat, mouse, pig, dog, cow, and horse. The organs destined for transplantation are maintained by an organ storage solution comprising one siRNA oligonucleotide or multiple siRNA oligonucleotides as a cocktail. siRNA can access the donor organ and cells easily and selectively, which facilitates the reduction of potentially harmful systemic side effects.

In current practice, donor organs are subjected to flushing and storage in static or recirculating systems, in hypothermic conditions (less than 37° C. for humans, e.g. 4° C.) or normothermic conditions (37° C. for humans), in specially formulated solutions (organ preservation solutions) in order to wash out debris and to decrease damage during transportation. The methods of the present invention include siRNA transfection of the donor organ and cells during organ preservation. This is an attractive method, because siRNA applied ex vivo to the organ to be donated would not be administered systemically to organ recipients, and treatment could be delivered specifically to the site of inflammation. This method could be useful to prevent graft failure without systemic adverse effects.

The siRNA transfection formulation is used for flushing the solid donor organ in situ and/or ex vivo, and for static or machine perfusion organ storage. The formulated solution is useful for both local injection into the solid organ and to bathe the entire solid organ by submerging it in the siRNA formulation.

The siRNA agent can be used as either single or multiple duplexes, targeting single or multiple genes, with or without transfection carriers for the treatment of the transplanted organs (tissues) and cells. The transfection agents include but are not limited to synthetic polymers, liposomes and sugars, etc. The siRNA agents can also be used with other agents such as small molecule and monoclonal antibody inhibitors, immune modulators and other types of oligonucleotides. The injection of and submerging of organs for transplantation with the siRNA/carrier solution will minimize tissue damage and host rejection, and therefore, will enhance the success of the transplanted organ in terms of organ function and survival and the minimization of co-morbidities.

Also in the present invention, various organs and cells can be treated by siRNA/carrier formulation during the process of transplantation. All solid organ transplantations essentially require surgical preparation of the donor, which may include flush perfusion of the body, or of specific organs to be used in transplantation. Perfusion may be with one or more fluids. The organ(s) are removed for storage during transportation to the recipient, and the organ is surgically implanted into the recipient. Organs useful in the methods of the invention include, but are not limited to, kidney, liver, heart, pancreas, pancreatic islets, small bowel, lung, cornea, limb, and skin, as well as cells in culture corresponding to each of those organs. One example, hepatocyte cell lines, are beginning to be developed as universal donors for isolated liver cell transplantation, which is a less invasive method than orthotopic liver transplantation for treatment of metabolic liver disease. Costimulation via pathways such as CD28/B7 or CD40/CD40L is a major concern for the success of such transplantation (2). Therefore, using siRNA/carrier formulation to silence both CD28 or CD40 pathways will be a good strategy to improve the success rate of the transplant.

Another example for renal transplant failure is the infection of parvovirus B19 (PV-B19) after solid organ transplantation which may cause pure red cell aplasia (PRCA). PV-B19 infection in immunosuppressed transplant recipients is associated with significant morbidity (1). Using siRNA to inhibit PV-B19 or any other viral infection and replication is an adjunct therapy for improvement in renal transplant by treatment of both donor organ and transplant recipient during the initial phase of the transplantation.

In another of its aspects, the present invention provides compositions comprising one or more siRNA duplexes in which siRNA can simultaneously target several genes involved in allograft or xenograft rejection or ischemia/reperfusion injury. A combination of multiple siRNA duplexes could be more effective for inhibition of allograft rejection or ischemia/reperfusion injury.

The process of immune modulation offers a plethora of molecular targets for siRNA silencing using the methods of the invention such as (1) molecules on lymphocytes associated with activation; (2) molecules on antigen presenting cells (APCs) which stimulate lymphocytes such as MHC class II and costimulatory molecules; (3) soluble molecular signals such as cytokines such as TNF-α, IFN-β, IL-1, IL-6, IL-8; (4) molecules associated with lymphocyte extravasation and homing such as Vascular Cell Adhesion Molecule-1, Intercellular Adhesion Molecular-1; and (5) effector molecules of immunity such as but not limited to complement factor C3. Additional candidate target genes include Intercellular Adhesion Molecule-1, Major Histocompatibility Complex Class I, Major Histocompatibility Complex Class II, IFN-γ, CD80, CD86, CD40 and CD40L.

The present invention also provides methods and compositions for using siRNA oligo cocktail (siRNA-OC) as therapeutic agent useful in the methods of the invention or to achieve more potent antiangiogenesis efficacy for treatment of cancer and inflammations. This siRNA oligo cocktail comprises at least three duplexes targeting at least three mRNA targets. The siRNA oligo cocktail may comprise any of the siRNA sequences listed in tables 1-15. In one embodiment, the siRNA oligo cocktail comprises the siRNAs specific for complement C3, MHC-II, and IFNγ. The present invention is based on two important aspects: first, the siRNA duplex is a very potent gene expression inhibitor, and each siRNA molecule is made of short double-stranded RNA oligo (21-23 nt, or 24-25 nt, or 26-29 nt) with the same chemistry property; Second, allograft or xenograft rejection and ischemia/reperfusion injury relate, in part, to overexpressions of endogenous genes. Therefore, using siRNA-OC targeting multiple genes represents an advantageous therapeutic approach, due to the chemical uniformity of siRNA duplexes and synergistic effect from down regulation of multiple disease- or injury-causing genes. The invention defines that siRNA-OC is a combination of siRNA duplexes targeting at lease three genes, at various proportions, at various physical forms, and being applied through the same route at the same time, or different route and time into disease tissues.

The siRNA-mediated silencing can be applied with either single siRNA targeting one such gene or a combination of multiple siRNAs targeting several target sequences within the same gene, or targeting various genes from different categories such as those identified in this paragraph. For example, a composition comprising multiple siRNA duplexes may have each present with the same or different ratios. Thus, in a mixture of three siRNAs duplex I, duplex II and duplex III may either each be present at 33.3% (w/w) of total siRNA agent each, or at 20%, 45% and 35% respectively, by way of nonlimiting example.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. The materials, methods and examples are illustrative only and not intended to be limiting.

### Example 1

### siRNA Mediated C3 Expression Knockdown In Vitro

RNA interference blocks gene expression according to small unique segments of their sequence. This natural process can be exploited to reduce transcription of specific genes. In transplantation, it is established that donor derived complement C3 is rapidly upregulated in ischemia/reperfusion injury (I/RI), contributing to tissue damage. Complement C3 is described as a local mediator of various forms of injury and immune regulation and is a valid target for gene knockdown after transplant ischemia/reperfusion injury that may well assist in the regulation of allo-immunity as well. This study sought to exploit siRNA to knock-down C3 gene expression in donor organs.

Rat renal epithelial cell lines were stimulated with 10 μg/ml IL-1 and 0.1 μg/ml IL-6 to upregulate C3 gene expression. 72 hours after stimulation, the cells were transfected with one of a panel of C3-specific siRNAs.

| | siRNA sequence |
|---|---|
| Sequence i.d. | (SEQ ID NOS 335-337) |
| C3-1 | CTG GCT CAA CGA CGA AAG ATA |
| C3-2 | CAC GGT AAG CAC CAA GAA GGA |
| C3-3 | AAG GGT GGA ACT GTT GCA TAA |

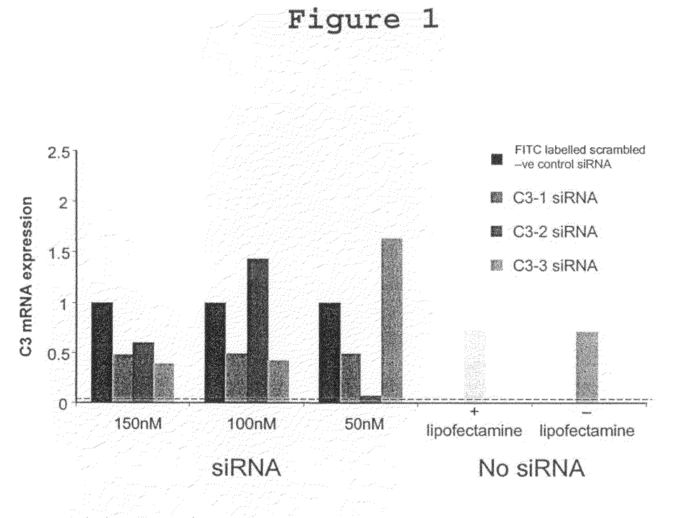

After 48 hours, C3 expression was determined by Real Time PCR. Results showed that C3 expression was upregulated in non-transfected cells after stimulation (FIG. 1). Cells treated with siRNA showed up to a 60% reduction of C3 expression as compared to control cells that were not treated with siRNA. These experiments identified the most effective C3 siRNA sequence from the panel that did not non-specifically induce IFNγ upregulation, a potential off-target effect of siRNA (labelled as C3-3 siRNA in FIG. 1).

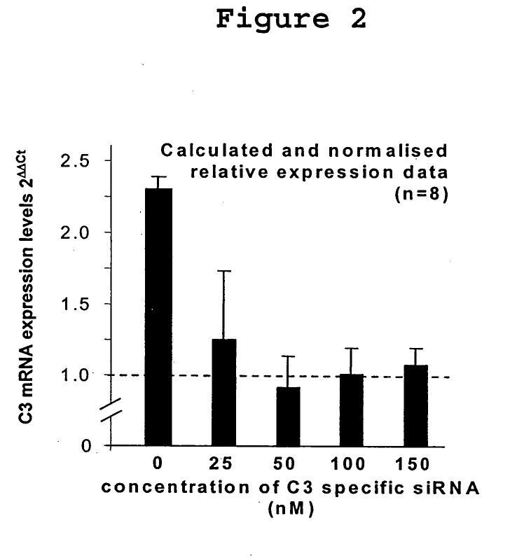

The candidate C3 siRNA obtained in the previous experiment was transfected into rat renal epithelial cells stimulated to express C3, as described above. A range of concentrations of this C3 specific siRNA produced significant (P<0.05) C3 mRNA knockdown, as measured by Real Time PCR (FIG. 2). This experiment demonstrates technical feasibility and efficacy of the C3 siRNA sequence identified for in vivo testing.

### Example 2

### siRNA Mediated C3 Expression Knockdown In Vivo

The most effective C3 siRNA, as determined in the previous experiment, was then packaged into synthetic polycationic nanoparticles that facilitate in vivo siRNA transfection. The nanoparticles are composed of PolyTran, a family of branched histidine (H) and lysine (K) polymers, effective for in vitro, in vivo, and ex vivo siRNA transfer. Their core sequence is as follows: R-KR-KR-KR (SEQ ID NO: 338), where R=[HHHKHHHKHHHKHHH]2 KH4NH4 (SEQ ID NO: 339). For in vivo experiments, the following branched HK polymers were initially tested for their efficacy to deliver siRNA into allograft cells: H3K4b. This branched polymer has the same core and structure described above except the R branches differ: R=KHHHKHHHKHHHKHHHK (SEQ ID NO: 340). The polymers were selected because of their in vitro or in vivo efficacy for different nucleic acid forms. The branched HK polymer was dissolved in aqueous solution and then mixed with siRNA aqueous solution at the listed ratios by mass, forming nanoparticles of average size of 150-200 nm in diameter. The HKP-siRNA aqueous solutions were semi-transparent without noticeable aggregation of precipitate. These solutions can be stored at 4° C. for at least three months.

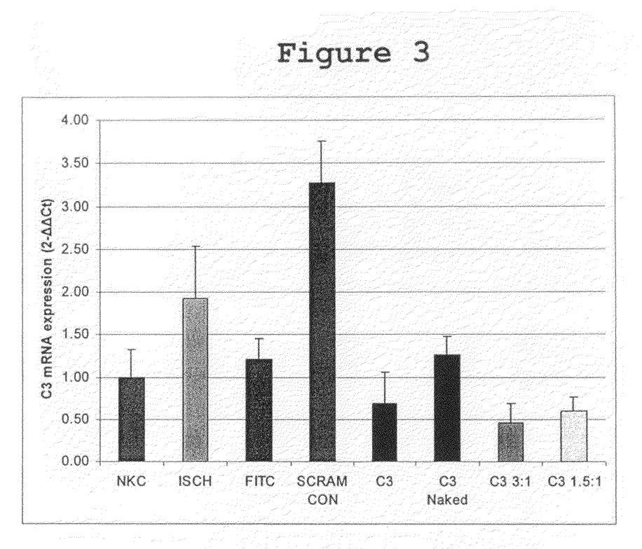

The nanoparticles were added to Hyper Osmolar Citrate perfusion fluid and administered to donor rat kidneys. After 4 hours of cold ischemia, the kidneys were transplanted into syngeneic hosts. Two days later the kidneys were harvested and C3 gene expression was determined by Real-Time PCR. Non-transplanted, non-treated kidneys served as a negative control (labelled NKC in FIG. 3), while perfused, transplanted kidneys not treated with siRNA served as a positive control (labelled as ISCH in FIG. 3). The levels in the siRNA-treated kidneys were normalized to mRNA levels in non-transplanted, non-treated kidneys. Results are shown in FIG. 3.

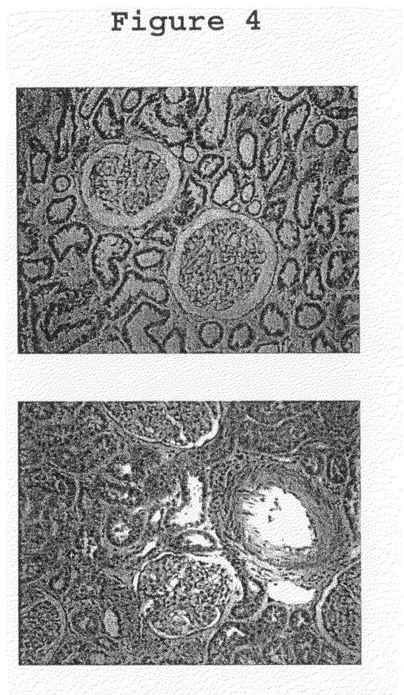

Results demonstrate that C3-siRNA reduced post-transplant C3 gene expression by 62.56% (P<0.05, n=4) compared to untreated transplants, to a level below that detected in-normal kidney. When compared against scrambled-FITC labelled siRNA control, C3 gene expression was reduced by 73.34% (P<0.05, n=4). The FITC-labelled scrambled siRNA controls exhibited a greater upregulation of C3 gene expression than the untreated kidneys, suggestive of off-target effects. Histology showed sparing from ischemia/reperfusion injury (I/RI) in kidneys treated with C3 siRNA before transplantation (FIG. 4), but direct fluorescence microscopy of cells and tissues perfused with FITC-labelled scrambled siRNA did not contain any detectable siRNA in tissues.

In conclusion, siRNA inhibition of C3 gene expression effectively reduced local C3 activity compared to controls. The nanoparticle strategy appears to overcome the problem of effective siRNA delivery. It now appears possible to develop arrays of specific siRNA to diminish pro-inflammatory gene expression in donor organs as adjunct therapies to conventional immunosuppression or tolerance induction.

### Example 3

### Determination of Peptide Sequences Concentrated in Transplanted Kidneys by Phage Display

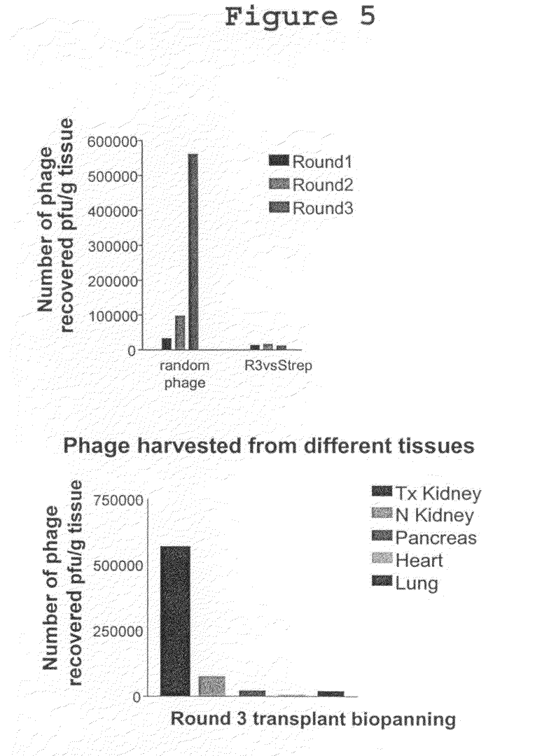

In order to provide organ target specificity for siRNA-containing nanoparticles, peptides concentrated in the organ of interest can be identified by phage display. This method was used to identify candidate target peptides in the rat model of kidney transplantation described above. Donor kidneys were flushed with Hyper Osmolar Citrate and stored at 4° C. for 4 hours before transplantation into a syngeneic host. After 48 hours, recipients were anaesthetized and injected via the tail vein with the prepared cysteine-constrained 7 mer phage library (New England Biolabs). After 5 minutes, the transplanted kidneys were harvested and phage extracted from the kidney, in a first round of “in vivo biopanning”. The extracted phage were expanded in E. coli bacteria before being injected into another kidney transplant recipient. This biopanning was repeated for a total of three rounds. After each round, a sample of phage was taken to estimate the numbers present in the transplanted kidney. After each expansion, a sample of phage was grown in bacterial colonies on agar plates so that phage could be isolated and the DNA sequence of the expressed library peptide could be determined. FIG. 5 (lower panel) shows increasing numbers of phage retrieved from transplanted kidneys after each round of biopanning (random phage), as compared to a control targeting streptavidin (R3vsStrep). Examples of identified peptide sequences concentrated in the kidney are C-LPSPKRT-C (SEQ ID NO: 341), C-LPSPKKT-C (SEQ ID NO: 342), C-PTSVPKT-C (SEQ ID NO: 343). After the third round of biopanning, phage are concentrated in the transplanted kidney and are found in much lower numbers in other organs of the recipient (FIG. 5, lower panel). The candidate peptides can be incorporated into TargeTran nanoparticles to provide specificity for siRNA targeting to transplanted organs.

### LITERATURE

1. Subtirelu M M et al. Acute renal failure in a pediatric kidney allograft recipient treated with intravenous immunoglobulin for parvovirus B19 induced pure red cell aplasia. Pediatr Transplant. 2005 December; 9(6):801-4.
2. Sampietro R, et al. Extension of the adult hepatic allograft pool using split liver transplantation. Acta Gastroenterol Belg. 2005 July-September; 68(3):369-75.
3. Chalermskulrat W, et al. Combined donor-specific transfusion and anti-CD154 therapy achieves airway allograft tolerance. Thorax. 2005 Oct. 27; [Epub ahead of print].
4. Oliveira J G, et al. Humoral immune response after kidney transplantation is enhanced by acute rejection and urological obstruction and is down-regulated by mycophenolate mofetil treatment. Transpl Int. 2005 November; 18(11):1286-91.
5. McManus, M. T. and P. A. Sharp (2002) Gene silencing in mammals by small interfering RNAs. Nature Review, Genetics. 3(10):737-747.
6. Lu, P. Y. et al. (2003) siRNA-mediated antitumorigenesis for drug target validation and therapeutics. Current opinion in Molecular Therapeutics. 5(3):225-234.
7. Lu, P. Y. et al (2-002) Tumor inhibition by RNAi-mediated VEGF and VEGFR2 down regulation in xenograft models. Cancer Gene Therapy. 10 (Supplement)) S4.
8. Kim, B. et al. (2004) Inhibition of ocular angiogenesis by siRNA targeting vascular endothelial growth factor-pathway genes; therapeutic strategy for herpetic stromal keratitis. Am. J. Pathol. 165 (6): 2177-85.
9. Lu, P. Y. and M. Woodle (2005) Delivering siRNA in vivo For functional genomics can novel therapeutics. In RNA Interference Technology. Cambridge University Press. P 303-317.
10. Lu, P. Y. et al. (2005) Modulation of angiogenesis with siRNA inhibitors for novel therapeutics. TRENDS in Molecular Medicine. 11(3), 104-13.

1-45. (canceled)

46. A targeting polynucleotide molecule, wherein the targeting polynucleotide molecule is double-stranded and comprises an antisense strand and a sense strand, wherein the antisense strand consists of a complement of a sequence selected from the group consisting of SEQ ID NOs: 55-64, 90-99, 110-119, 130-139, 150-159, 169-174, 185-194, 205-214, 225-234, 245-254, 265-274, 285-294, 305-314 and 325-334, optionally with an overhang of one to four nucleotides; and wherein the sense strand consists of a complement of the antisense strand, optionally with an overhang of one to four nucleotides.

47. The targeting polynucleotide of claim 46 that is a 25 nucleotide, blunt-ended double-stranded short interfering RNA (siRNA).

48. The targeting polynucleotide of claim 46, comprising at least one nucleotide that is modified.

49. A composition comprising the targeting polynucleotide of claim 46 and a carrier.

50. The composition of claim 49, further comprising one or more additional nucleic acid molecules that induce RNA interference and decrease the expression of a gene of interest.

51. The composition of claim 50, wherein at least one of the one or more additional nucleic acid molecules decreases the expression of an immunomodulatory or an immunoeffector gene.

52. The composition of claim 51, wherein the immunomodulatory or immunoeffector gene is selected from the group consisting of: C3 (complement C3), ICAM1 (Intercellular Adhesion Molecule-1), VCAM-1 (Vascular Cell Adhesion Molecule-1), IFN-γ (Interferon gamma), IL-1 (Interleukin-1), IL-6 (Interleukin-6), IL-8 (Interleukin-8), TNF-α (Tumor necrosis factor-alpha), CD80, CD86, MHC-II (Major Histocompatibility Complex Class II), MHC-I (Major Histocompatibilty Complex Class I), CD28, CTLA-4 and PV-B19.

53. The composition of claim 49, wherein the carrier is synthetic.

54. The composition of claim 53, wherein the synthetic carrier comprises a cationic polymer-nucleic acid complex.

55. The composition of claim 54, wherein the cationic polymer is a histidine-lysine co-polymer.

56. The composition of claim 53, wherein the synthetic carrier further comprises a hydrophilic component.

57. The composition of claim 56, wherein the hydrophilic component comprises polyethylene glycol or a polyacetal, or any combination thereof.

58. The composition of claim 53, wherein the synthetic carrier further comprises a targeting ligand.

59. The composition of claim 49, comprising an additional therapeutic agent.

60. A method for reducing the protein level of a gene selected from ICAM1, VCAM-1, IFN-γ, IL-1, IL-6, IL-8, TNF-α, CD80, CD86, MHC-II, MHC-I, CD28, CTLA-4 and PV-B19 in a cell, comprising introducing into the cell the targeting polynucleotide molecule of claim 46.

61. A targeting polynucleotide molecule, wherein the targeting polynucleotide molecule is double-stranded and comprises an antisense strand and a sense strand, wherein the antisense strand consists of a complement of a sequence selected from the group consisting of SEQ ID NOs: 26-35, optionally with an overhang of one to four nucleotides; and wherein the sense strand consists of a complement of the antisense strand, optionally with an overhang of one to four nucleotides.

62. A composition comprising the targeting polynucleotide of claim 61 and a carrier.

63. The composition of claim 62 further comprising the targeting polynucleotide of claim 46.

64. A method for reducing the C3 protein level in a cell, comprising introducing into the cell the targeting polynucleotide of claim 61.

65. A method for suppressing rejection of a transplanted organ by a recipient of the organ, comprising the step of contacting the organ with the targeting polynucleotide of claim 61 before transplanting the organ into the recipient.

66. A method for suppressing rejection of a transplanted organ by a recipient of the organ, comprising the step of contacting the organ with the composition of claim 62 before transplanting the organ into the recipient.

67. A method for suppressing rejection of a transplanted organ by a recipient of the organ, comprising the step of contacting the organ with the composition of claim 63 before transplanting the organ into the recipient.
